SEQUENCE LISTING (2 sequences)

SEQ ID NO: 1 - protein, 566 aa, Influenza A virus
MKTIIALSYIFCLPLGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 2 - protein, 566 aa, Influenza A virus
MKTIIALSYMFCLVFAQNLPGNDNSTATLCLGHHAVPNGTLVKTITNDQIEVTNATELVQSSSTGRICDSPHRILDGKNCTLIDALLGDPHCDGFQNEKWDLFVERSKAFSNCYPYDVPDYASLRSLVASSGTLEFINEGFNWTGVTQSGGSYTCKRGSNNSFFSRLNWLYESESKYPVLNVTMPNNGKFDKLYIWGIHHPSTDKEQTNLYIRASGRVTVSTKRSQQTVIPNIGSRPWVRGLSSRISIYWTIVKPGDILLINSTGNLIAPRGYFKIRTGKSSIMRSDAPIGTCSSECITPNGSIPNDKPFQNVNKITYGACPRYVKQNTLKLATGMRNVPEKQTRGIFGAIAGFIENGWEGMVDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

(19) United States
(12) Patent Application Publication
(10) Pub. No.: US 2024/0189415 A1
(43) Pub. Date: Jun. 13, 2024

(54) GRAPHENE OXIDE NANOPARTICLES AND METHODS OF USE FOR STIMULATING IMMUNE RESPONSES

(71) Applicant: GEORGIA STATE UNIVERSITY RESEARCH FOUNDATION, INC., Atlanta, GA (US)

(72) Inventors: Baozhong Wang, Duluth, GA (US); Chunhong Dong, Atlanta, GA (US)

(21) Appl. No.: 18/286,191

(22) PCT Filed: Apr. 8, 2022
(86) PCT No.: PCT/US2022/024059

§ 371 Date: Oct. 9, 2023

Related U.S. Application Data
(60) Provisional application No. 63/172,628, filed on Apr. 8, 2021.

(51) Int. Cl. A61K 39/145; A61K 39/00; A61P 31/16; C12N 7/00

(52) U.S. Cl. CPC A61K 39/145; A61P 31/16; C12N 7/00; A61K 2039/55555; A61K 2039/55561; A61K 2039/575; C12N 2760/16134; C12N 2760/16171

(57) ABSTRACT

Disclosed are functionalized graphene oxide nanoparticles and methods their use in inhibiting and treating microbial infection. The disclosure herein further provides for methods of making functionalized graphene oxide nanoparticles and said nanoparticles further incorporating microbial antigens for inducing antigen specific immune responses.

This application claims the benefit of U.S. Provisional Application No. 63/172,628, filed on Apr. 8, 2021, which in incorporated herein by reference in its entirety.

This invention was made with government support under Grant No. AI101047, AI116835, and AI143844 awarded by the National Institutes of Health. The government has certain rights in the invention.

### I. BACKGROUND

Influenza remains one of the leading infectious diseases causing morbidity and mortality worldwide. Vaccination is the most cost-effective approach to preventing influenza virus infection. However, current virus-based seasonal influenza vaccines induce strain-specific immunity and are less effective against mismatched strains that may cause influenza epidemics. Furthermore, there is no vaccine countermeasure available for new pandemic strains. Intranasal (i.n.) immunization is a promising vaccination route for infectious respiratory diseases, such as influenza. This vaccination route can induce both systemic and mucosal immune responses. Secretory immunoglobulin A (sIgA) and immunoglobulin G (IgG) may prevent influenza infection at the portal of virus entry. Influenza mucosal immunity has been reported to confer cross-protection against heterologous and heterosubtypic viruses.

However, the development of i.n. influenza vaccines has progressed slowly. The cold-adapted live-attenuated influenza virus (LAIV) vaccine is the only available human i.n. influenza vaccine up to date. Studies have shown that the LAIV vaccine could provide heterologous immunity. Nevertheless, safety concerns of LAIV are raised, especially in high-risk populations, such as infants under 2-year-old and the elderly over 50. LAIV could undergo genetic reassortments and revert into a virulent form, thus posing a risk. Besides, the suboptimal protective efficacy of LAIV vaccines was reported in children in the 2009 H1N1 pandemic. A new generation of virus-independent, safe, and efficient influenza intranasal vaccines that induce broader cross-protection with high efficacy is urgently needed.

### II. SUMMARY

Disclosed are methods and compositions related to functionalized graphene oxide nanoparticles.

In one aspect, disclosed herein are compositions comprising i) a functionalized graphene oxide (GO) nanoparticle (including, but not limited to GO nanoparticle sheets) comprising polyethyleneimine (PEI) (GO-PEI, GP) and ii) a microbial antigen (such as for example, peptides, polypeptides, proteins, inactivated viruses or bacteria, and heat killed viruses), vaccines (including, but not limited to intranasally administered vaccines), and/or pharmaceutical agents (including, but not limited to intranasally administered pharmaceutical agents). In some aspects, the functionalized GO nanoparticle can be pegylated.

For example, disclosed herein are compositions comprising a functionalized graphene oxide (GO) nanoparticle (including, but not limited to GO nanoparticle sheets) comprising polyethyleneimine (PEI) (GO-PEI, GP) and ii) a microbial antigen wherein the microbial antigen comprises a viral antigen from a virus selected from the group consisting of Herpes Simplex virus-1 (such as, for example, glycoprotein D and/or glycoprotein G), Herpes Simplex virus-2 (such as, for example, glycoprotein D and/or glycoprotein G), Varicella-Zoster virus (such as, for example, glycoprotein E), Epstein-Barr virus (such as, for example the EBV glycoprotein), Cytomegalovirus (such as, for example the CMV glycoprotein), Human Herpes virus-6, Variola virus, Vesicular stomatitis virus, Hepatitis A virus, Hepatitis B virus (including, but not limited to the hepatitis B virus surface antigen), Hepatitis C virus (such as, for example, the Hepatitis C E1, E2, or E3 proteins), Hepatitis D virus, Hepatitis E virus, Rhinovirus, Coronavirus (including, but not limited to spike or envelope proteins from avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A (such as, for example the hemagglutinin (HA) protein including the HA1 and HA2 protein and including trimeric HA), Influenza virus B (such as, for example the hemagglutinin (HA) protein including the HA1 and HA2 protein and including trimeric HA), Measles virus (such as, for example the hemagglutinin protein), Polyomavirus, Human Papilomavirus, Respiratory syncytial virus (such as, for example the RSV G protein), Adenovirus, Coxsackie virus, Dengue virus (such as, for example capsid protein, envelope protein, and/or premembrane/membrane protein), Mumps virus, Poliovirus, Rabies virus (including, but not limited to the Rabies glycoprotein), Rous sarcoma virus, Reovirus, Yellow fever virus, Zika virus (such as, for example capsid protein, envelope protein, and/or premembrane/membrane protein), Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A (including, but not limited to viral protein 4 and viral protein 7), Rotavirus B (including, but not limited to viral protein 4 and viral protein 7), Rotavirus C (including, but not limited to viral protein 4 and viral protein 7), Sindbis virus, Simian Immunodeficiency virus, Human T-cell Leukemia virus type-1, Hantavirus, Rubella virus, Simian Immunodeficiency virus, Human Immunodeficiency virus type-1 (such as, for example, glycoprotein (gp), envelope protein (Env), or gag protein), and Human Immunodeficiency virus type-2.

Also disclosed herein are compositions comprising i) a functionalized graphene oxide (GO) nanoparticle (including, but not limited to GO nanoparticle sheets) comprising polyethyleneimine (PEI) (GO-PEI, GP) and ii) a microbial antigen of any preceding aspect, wherein the microbial antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

In one aspect, disclosed herein are compositions comprising i) a functionalized graphene oxide (GO) nanoparticle (including, but not limited to GO nanoparticle sheets) comprising polyethyleneimine (PEI) (GO-PEI, GP) and ii) a vaccine of any preceding aspect, wherein the comprises a heat killed virus or inactivated virus, including, but not limited to seasonal flu vaccines, and coronavirus vaccines.

Also disclosed herein are compositions comprising i) a functionalized graphene oxide (GO) nanoparticle (including, but not limited to GO nanoparticle sheets) comprising polyethyleneimine (PEI) (GO-PEI, GP) and ii) pharmaceutical agent of any preceding aspect, wherein the pharmaceutical agent comprises zanamivir, oseltamivir, peramivir, baloxavir, midazolam, lorazepam, flumazenil, dexmedetomidine, ketamine, fentanyl, hydromorphone, butorphanol, naloxone, insulin, fluticasone, ciclesonide, budesonide, dupilumab, mometasone, albuterol, reslizumab, zileuton, mepolizumab, omalizumab, and haloperidol.

In some aspects, disclosed herein are compositions comprising i) a functionalized graphene oxide (GO) nanoparticle (including, but not limited to GO nanoparticle sheets) comprising polyethyleneimine (PEI) (GO-PEI, GP) and ii) a microbial antigen, vaccine, and/or pharmaceutical agent of any preceding aspect, wherein the weight-to-weight ratio of GO-PEI particles to microbial antigen ranging from 10:1 to 1:10, for example, 10:1, 8:1, 6:1, 5:1, 4:1, 3:1, 2:1, 1:1, 1:2, 1:4, 1:5, 1:6, 1:8, or 1:10.

Also disclosed herein are compositions comprising i) a functionalized graphene oxide (GO) nanoparticle (including, but not limited to GO nanoparticle sheets) comprising polyethyleneimine (PEI) (GO-PEI, GP) and ii) microbial antigen, vaccine, and/or pharmaceutical agent of any preceding aspect, further comprising an adjuvant (such as, for example, CpG oligonucleotide (CpG ODN)). In some aspects, the weight-to-weight ratio of GO-PEI particles to microbial antigen to adjuvant comprises 10:5:2.5 or 10:5:1.

In some aspects, disclosed herein are compositions comprising i) a functionalized graphene oxide (GO) nanoparticle (including, but not limited to GO nanoparticle sheets) comprising polyethyleneimine (PEI) (GO-PEI, GP) and ii) a microbial antigen, vaccine, and/or pharmaceutical agent of any preceding aspect, wherein the combination of a functionalized GO nanoparticle, microbial antigen with or without an adjuvant has a diameter ranging from 50 nm to 300 nm, preferably from about 160 nm to 200 nm. For example, the combination of the GO nanoparticle and microbial antigen with or without an adjuvant has a diameter of about 50, 60, 70, 80, 90, 100, 110, 120, 130, 140, 150, 160, 170, 180, 190, 200, 210, 220, 230, 240, 250, 260, 270, 280, 290, or 300 nm.

Also disclosed herein are compositions comprising i) a functionalized graphene oxide (GO) nanoparticle (including, but not limited to GO nanoparticle sheets) comprising polyethyleneimine (PEI) (GO-PEI, GP) and ii) a microbial antigen, vaccine, and/or pharmaceutical agent of any preceding aspect, wherein the composition has tunable zeta potential. In some aspects the composition has a zeta potential greater than 30 mV.

In one aspect, disclosed herein are vaccines comprising the compositions comprising i) a functionalized graphene oxide (GO) nanoparticle (including, but not limited to GO nanoparticle sheets) comprising polyethyleneimine (PEI) (GO-PEI, GP) and ii) a microbial antigen, vaccine, and/or pharmaceutical agent of any preceding aspect.

Also disclosed herein are methods of treating, inhibiting, reducing, decreasing, ameliorating and/or preventing a microbial infection comprising administering to a subject at risk of being infected or that is infected with a microbe, the vaccine of any preceding aspect or any of the compositions comprising i) a functionalized graphene oxide (GO) nanoparticle (including, but not limited to GO nanoparticle sheets) comprising polyethyleneimine (PEI) (GO-PEI, GP) and ii) a microbial antigen or vaccine of any preceding aspect.

In one aspect, disclosed herein are methods of inducing an immune response (such as, for example, mucosal immune responses and/or production of microbial specific IgA antibodies, microbial specific IgG antibodies, IL-6 production, TNF-α production) in a subject to a microbial antigen, vaccine, and/or pharmaceutic agent comprising administering to the subject the vaccine of any preceding aspect or any of the compositions comprising i) a functionalized graphene oxide (GO) nanoparticle (including, but not limited to GO nanoparticle sheets) comprising polyethyleneimine (PEI) (GO-PEI, GP) and ii) a microbial antigen of any preceding aspect.

Also disclosed herein are methods of delivering an antigen, vaccine, and/or pharmaceutical agent to a mucosal surface of a subject comprising administering to a subject at risk of being infected with a microbe the vaccine of any preceding aspect or any of the compositions comprising i) a functionalized graphene oxide (GO) nanoparticle (including, but not limited to GO nanoparticle sheets) comprising polyethyleneimine (PEI) (GO-PEI, GP) and ii) a microbial antigen of any preceding aspect.

In one aspect, disclosed herein are methods of making a vaccine to a microbial antigen comprising a) obtaining a graphene oxide (GO) powder; b) sonicating GO flakes in an ice bath until the GO particles are less than 500 nm in diameter (such as for example, less than or equal to 200 nm, between 160-200 nm, between 160 and 170 nm) using tip ultrasonication; c) adding polyethyleneimine (PEI) and sonicating; d) activating the GO-PEI with EDC; and e) adding the microbial antigen to the activated GO-PEI particles at a GO-PEI to Ag weight-to-weight ratio from 10:1 to 1:10, for example, 10:1, 8:1, 6:1, 5:1, 4:1, 3:1, 2:1, 1:1, 1:2, 1:4, 1:5, 1:6, 1:8, or 1:10

Also disclosed herein are methods of making a vaccine to a microbial antigen of any preceding aspect, further comprising co-loading an adjuvant (such as, for example, CpG ODN) with the microbial antigen onto the GO-PEI nanoparticles. In some aspects, the weight-to-weight ratio of GO-PEI particles to microbial antigen to adjuvant comprises 10:5:2.5 or 10:5:1.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A and 1B show a schematic illustration of the preparation and performance of influenza GO-PEI (GP) nanoparticles. FIG. 1A shows preparation of GP and GP-HA/adjuvant nanoparticles. FIG. 1B shows immunoenhancing effects of GP nanoparticle vaccines. GP nanoparticle vaccines showed enhanced cellular uptake in dendritic cells (DCs) and promoted inflammatory cytokine secretion and DC maturation. Intranasal (i.n.) vaccination with influenza GP nanoparticles induced significantly enhanced and cross-reactive immune protection against homologous and heterologous influenza virus challenges. sIgA, secretory IgA; CTL, cytotoxic T lymphocyte; Aic, Aichi virus; Phi, Philippines virus; SR, survival rate.

FIGS. 2A, 2B, and 2C show characterization of GO nanoparticles. FIG. 2A shows size changes of GO nanoparticles after different ultrasonication time. 1 mg/mL of pure GO powder was dispersed in distilled water. The sonication amplitude was set as 100 W, with 5 s pulse on and 5 s pulse off FIG. 2B shows an AFM image of GO nanoparticles. FIG. 2C shows the thermo-gravimetric analysis (TGA) spectra of GO and GO-PEI (GP) nanoparticles. The weight losses in the range of 250-400° C. correspond to the pyrolysis of the residual oxygen-containing groups and bonded PEI molecules. The conjugated PEI was around 17.94% after subtracting the proportion of the residual oxygen-containing groups in GO.

FIGS. 3A, 3B, 3C, 6D, 3E, 3F, 3G, 3H, 3I, and 3J show characterization of the influenza GP nanoparticles. FIG. 3A shows TEM image of GO nanoparticles. FIG. 3B shows Coomassie blue staining (CB) and Western blotting (WB) analysis. HA was detected with anti-Aichi HA antibodies. FIGS. 3C and 3D show nanoparticle size and Zeta-potential analysis. FIG. 3E shows antigen internalization by dendritic cells. JAWS II cells were treated with soluble Aichi HA (H3) or GP-H3 nanoparticles for 16 h at an H3 concentration of 10 μg/mL before capturing immunofluorescence images. Intracellular H3 was probed by fluorescent-labeled Ab (red). FIGS. 3F, 3G, and 3H show proinflammatory cytokine production. JAWS II cells were treated with different formulations at indicated H3 concentrations for 16 h. Then, TNF-α and IL-6 productions were assessed. FIGS. 3I and 3J show CD86 expression on JAWS II cells. After the anti-CD86 antibody staining, cells were analyzed using flow cytometry. MFI, mean fluorescence intensity. Data are presented as mean±SEM. Statistical significance was analyzed by One-Way ANOVA followed by Dunnett's multiple comparison test, comparing the mean of each group with the mean of the control group (Untreated). (*p<0.05; **p<0.01; ***p<0.001; ****p<0.0001; ns, p>0.05.)

FIGS. 4A, 4B, 4C, and 4D show characterization of purified H3 protein and constructed influenza GP nanoparticles. FIG. 4A shows an SDS-PAGE followed by Western blotting of purified H3 protein after BS3 (0, 0.5, 5, and 10 mM) crosslinking. FIG. 4B shows Coomassie blue staining analysis for the particle pellet and supernatants of GP-HA at different GP to HA ratios to evaluate the loading capability of Aichi HA proteins on GP nanoparticles. FIG. 4C shows the UV-Vis absorption spectra of soluble H3 protein, GO-PEI (GP), and GP-H3 nanoparticles. FIG. 4D shows the agarose gel electrophoresis result to determine the free CpG in the supernatant of (1) soluble CpG in PBS; (2) GP-H3/CpG (10:5:1); (3) GP-H3/CpG (10:5:2.5); and (4) H3+CpG mix after centrifugation at 15000 rpm for 20 minutes.

FIG. 5A shows immunofluorescence images of untreated JAWS II cells. Cells were fixed, permeabilized, and then treated with anti-Aichi virus serum and DyLight™ 649 anti-mouse IgG antibody. The very weak background fluorescence in untreated cells indicated little nonspecific adsorption of fluorescent antibodies.

FIG. 5B shows HAI antibody titers in nasal washes and BALF of vaccinated mice in different groups.

FIGS. 6A, 6B, 6C, 6D, 6E, 6F, 6G, and 6H show humoral immune responses. FIG. 6A shows a timeline of immunization, sample collection, and challenge experiments. BALB/c mice were i.n. immunized twice in a 4-wk interval. Groups included soluble H3, GP-H3, GP-H3/CpG, and H3+CpG mix. Naïve mice were used as controls. FIGS. 3B and 3C show Aichi virus-specific IgG endpoint titers in mice prime and boost sera, respectively. FIGS. 3D and 3E show HAI and neutralization titers in mouse boost sera. FIGS. 3F and 3G show OD values at 450 nm for diluted nasal washes and BALF samples to detect mucosal IgA by ELISA. FIG. 3H shows OD values at 450 nm for diluted BALF samples to detect mucosal IgG. Data are presented as mean±SEM. Statistical significance was analyzed by One-Way ANOVA followed by Dunnett's multiple comparison test, comparing the mean of each group with the mean of the H3 group. (*p<0.05; **p<0.01; ***p<0.001; ****p<0.0001; ns, p>0.05.)

FIGS. 7A, 7B, 7C, 7D, 7E, 7F, 7G, and 7H show cellular immune responses. Spleens and cervical lymph nodes (CLN) were harvested from mice 3 wks post boosting immunization to prepare single-cell suspensions. FIGS. 7A and 7B show IL-4-secreting cells in splenocytes and CLN cells. Cell cultures were stimulated with H3 (4 μg/mL). FIGS. 7C and 7D show antigen-specific IgG and IgA plasma cells in splenocytes. FIGS. 7E and 7F show CD3⁺CD4⁺ T cell proliferation by CFSE staining assay. FIGS. 7G and 7H show CD3⁺CD8⁺ T cell proliferation. The population with decreased fluorescence intensity of CFSE was labeled as P area, representing the cells that have undergone proliferation. CFSE: Carboxyfluorescein succinimidyl amino ester. Data are presented as mean±SEM. Statistical significance was analyzed by One-Way ANOVA followed by Dunnett's multiple comparison test, comparing the mean of each group with the mean of a control group (H3). (*p<0.05; **p<0.01; ***p<0.001; ****p<0.0001; ns, p>0.05.)

FIGS. 8A and 8B show IFN-γ secreting lymphocytes in spleens and CLNs of vaccinated mice.

FIG. 8C shows antigen-specific IgE antibody levels in sera of immunized mice.

FIG. 9A shows histological examination of mouse nasal mucosa by H&E staining one day post-vaccination. Bars represent 100 μm in length.

FIG. 9B shows mice body weight changes post-vaccination. Bar scales represent 200 μm in length.

FIG. 9C shows lung histological studies 7 days post-vaccination.

FIG. 9D shows the evaluation of Aichi virus-specific IgG and IgA plasma cells in NALT cells by ELISpot method.

FIGS. 10A and 10B show evaluation of CD3+CD4+ (10A) and CD3+CD8+ (10B) T cell proliferation capabilities using the CFSE staining assay. P area with decreased fluorescence intensity of CFSE represents the cells that have undergone proliferation.

FIGS. 11A and 11B show evaluation of the percentage of CD3+CD4+ (11A) and CD3+CD8+ (11B) T cell populations in splenocytes by flow cytometry.

FIGS. 12A, 12B, 12C, 12D, 12E, 12F, 12G, and 12H show protective efficacy against homologous influenza virus challenge. Mice were challenged with 15×LD₅₀ of mouse-adapted Aichi (Aic) virus 4 wks post boosting immunization. FIGS. 12A and 12B show morbidity (12A) and mortality (12B) of mice after challenge. FIG. 12C shows histological pathology analysis by H&E staining. The uninfected mouse lung section was used as a negative control. Red arrows in images indicate leukocyte infiltration. Images are representatives from each group. Bars represent 200 μm in length. FIG. 12D shows a bar chart showing the scores of leukocyte infiltration degree. FIG. 12E shows determination of mouse lung virus titers. FIGS. 12F, 12G, and 12H show evaluation of inflammatory cytokine (TNF-α, IL-12, and IL-6) levels in BALF of infected mice. Data are presented as mean±SEM. Statistical significance was analyzed by One-Way ANOVA followed by Dunnett's multiple comparison test, comparing the mean of each group with the mean of a control group. The infected naïve mouse group was used as the control group in 12D and 12E, and the H3 group was used as the control group in 12F and 12H. (*p<0.05; **p<0.01; ***p<0.001; ****p<0.0001; ns, p>0.05.)

FIGS. 13A and 13B show body weight monitoring and antibody analysis post-infection. FIG. 13A shows body weight changes of H3 and GP-H3 immunized mice after challenge with 15×LD50 of homologous mouse-adapted Aichi viruses. BALB/c mice (n=5) were challenged at 4 wks post boosting immunization. FIG. 13B shows Aichi virus-specific IgA and IgG antibody analysis in BALF of Aichi virus-infected mice.

FIGS. 14A, 14B, 14C, 14D, 14E and 14F show protective efficacy against heterologous influenza virus challenge. Mice were challenged with 2×LD₅₀ of mouse-adapted Philippines (Phi) virus 4 wks post boosting immunization. FIGS. 14A and 14B show morbidity (14A) and mortality (14B) of mice after challenge. FIG. 14C shows cross-protective IgG endpoint titers against the Phi virus in mice boost sera. FIGS. 14D and 14E show Phi virus-specific IgG and IgA levels in BALF. FIG. 14F shows Phi virus-specific IgA levels in nasal washes. FIG. 14G shows serum hrHA3-specific antibody levels. FIGS. 14H and 14I show IL-4 and IFN-γ-secreting splenocytes under inactivated Phi virus stimulation. Data are presented as mean±SEM. Statistical significance was analyzed by One-Way ANOVA followed by Dunnett's multiple comparison test, comparing the mean of each group with the mean of the H3 group. (*p<0.05; **p<0.01; ***p<0.001; ****p<0.0001; ns, p>0.05.)

FIG. 15A shows body weight changes of H3 and GP-H3 immunized mice post-challenge with 2×LD₅₀ of heterologous mouse-adapted Phi viruses. BALB/c mice (n=5) were challenged at 4 wks post boosting immunization

FIG. 15B shows Phi virus-specific neutralization antibody titers in mouse boost sera.

FIG. 15C shows Phi virus-specific HAI antibody titers in sera and BALF of vaccinated mice.

FIG. 15D shows control Elisa assay using a nonrelated His-tagged SARS-CoV-2 spike protein receptor-binding domain (RBD) as the coating antigen.

FIGS. 16A, 16B, 16C, and 16D show cross-binding antibody levels in mouse sera and mucosal washes against Wis virus. (16A) Serum IgG. (16B) IgA in the nasal washes. (16C, 16D) IgA and IgG in BALF

FIGS. 17A, 17B, 17C, and 17D show cross-binding antibody levels in mouse sera and mucosal washes against the rSH virus. (17A) Serum IgG. (17B) IgA in the nasal washes. (17C, 17D) IgA and IgG in BALF.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular microbial antigen, graphene oxide nanoparticle, and/or functionalized graphene oxide (GO) nanoparticle comprising polyethyleneimine (PEI) (GO-PEI, GP) is disclosed and discussed and a number of modifications that can be made to a number of molecules including the microbial antigen, graphene oxide nanoparticle, and/or functionalized graphene oxide (GO) nanoparticle comprising polyethyleneimine (PEI) (GO-PEI, GP) are discussed, specifically contemplated is each and every combination and permutation of microbial antigen, graphene oxide nanoparticle, and/or functionalized graphene oxide (GO) nanoparticle comprising polyethyleneimine (PEI) (GO-PEI, GP) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Intranasal vaccination with recombinant protein/peptide-based vaccines is an attractive strategy with high safety. Purified protein/peptide antigens eliminate safety concerns in individuals with egg allergies, possess minimal side effects, and could be produced quickly and cost-effectively. However, soluble protein vaccines are poorly immunogenic by intranasal (i.n.) immunization due to the harsh and tolerogenic mucosal environment. The selection of appropriate formulations and adjuvants is crucial for successful i.n. vaccines. Nanoparticle vaccine platforms have been applied for i.n. vaccine development in recent years. Nanoparticles serve as antigen and adjuvant carriers and immunostimulants themselves to enhance immune responses. The immunoenhancing effects of various nanoparticles have been reported. However, most nanoparticle vaccines suffer from low antigen-loading capacity, complicated and lengthy preparation procedures, and structural complexity because of covalent conjugation.

Two-dimensional (2-D) graphene oxide (GO) nanoparticles are a great vaccine platform due to their extraordinary attributes. These features include the high aspect ratio and ultra-large surface area for high-density antigen association, wealthy chemical groups for flexible surface modification, and noncovalent antigen loading via electrostatic adsorption, hydrogen bond, and hydrophobic and π-π stacking interactions. Besides, GO nanoparticles themselves are biocompatible and nonimmunogenic. Various GO vaccine formulations were demonstrated to induce improved immune responses by activating immune cells or triggering innate signaling. However, most prior studies were limited to conventional routes with tumor antigens for cancer immunotherapies. Studies on GO-based influenza i.n. vaccines are lacking.

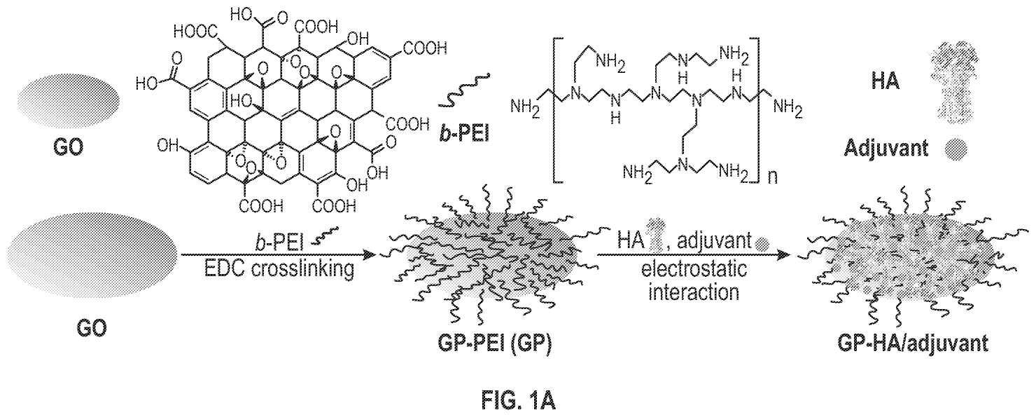

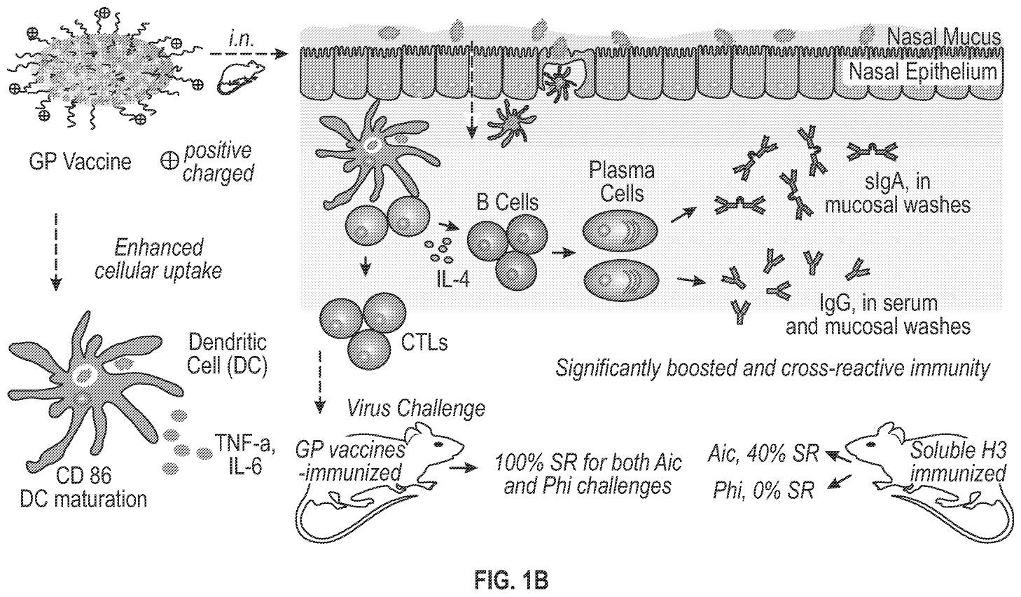

We developed a polyethyleneimine (PEI)-functionalized GO (GO-PEI, GP) influenza vaccine nanoplatform, prepared influenza GP nanoparticles by incorporating recombinant influenza HA, and investigated their immunoenhancing effects (FIG. 1). The work revealed that influenza GP nanoparticles enhanced antigen internalization and promoted the production of inflammatory cytokines and the maturation of JAWS II dendritic cells (DCs) during in vitro experiments. Intranasal vaccination with influenza GP nanoparticles induced robust humoral and cellular immune responses, conferring broader protection against both homologous and heterologous virus challenges in mice. Accordingly, in one aspect, disclosed herein are compositions comprising i) a functionalized graphene oxide (GO) nanoparticle comprising polyethyleneimine (PEI) (GO-PEI, GP) and ii) a microbial antigen (such as for example, peptides, polypeptides, proteins, inactivated viruses or bacteria, and heat killed viruses), vaccine (including, but not limited to intranasally administered vaccines such as, for example, seasonal flu vaccines and coronavirus vaccines), and/or pharmaceutical agent (such as, for example zanamivir, oseltamivir, peramivir, baloxavir, midazolam, lorazepam, flumazenil, dexmedetomidine, ketamine, fentanyl, hydromorphone, butorphanol, naloxone, insulin, fluticasone, ciclesonide, budesonide, dupilumab, mometasone, albuterol, reslizumab, zileuton, mepolizumab, omalizumab, and haloperidol). In some aspects, the functionalized graphene oxide (GO) nanoparticle can assemble into nanoparticle sheets.

We found that antigens in the outer shells of layered protein nanoparticles induced robust antibody responses while the antigens in the inner cores induced strong T cell responses. Therefore, we can generate new layered nanosheets by successive deposition of NP-M2e (as an inner layer) and hrHA (an outer layer) onto the new nanosheet scaffolds via electrostatic adsorption approach. A two-step process can prepare the nanosheet scaffolds. First, PEI will be conjugated to the carboxyl groups on GO via the Carbodiimide coupling method using N-(3-dimethyl aminopropyl-N′-ethyl carbodiimide) hydrochloride (EDC). Then, the GO-PEI nanosheets conjugate with PEG-NHS, designated as GPP nanosheets. Nanoparticle PEGylation can increase the stability and biocompatibility of the nanoparticles. Because of the charge shielding effect of PEG, PEGylation has become one of the most attractive strategies to improve the biocompatibility and physicochemical properties of diverse nanomedicines. PEGylated pulmonary surfactant-biomimetic nanoparticles have potentiated heterosubtypic influenza immunity of influenza vaccines and engineered mucus-penetrating drug carriers for sustained drug delivery at mucosal sites. The surface chemistry and storage stability of GPP nanosheets can be adjusted as needed by tuning the amount of conjugated PEG. The high versatility and flexibility of the GPP nanosheet scaffolds allow the co-incorporation of NP-M2e and hrHA successively to generate layered protein coatings, designed as hrHA/NP-M2e (outer layer/inner layer) Nano. Because of the GPP scaffold surface's high protein-loading capacity, the antigenic protein incorporation is nearly 100% efficient at scaffold to protein ratio from 1:2 to 2:1 at 4° C. overnight. Accordingly, in one aspect, disclosed herein are compositions comprising i) a functionalized graphene oxide (GO) nanoparticle (including, but not limited to nanoparticle sheets) comprising polyethyleneimine (PEI) (GO-PEI, GP) and ii) a microbial antigen (such as for example, peptides, polypeptides, proteins, inactivated viruses or bacteria, and heat killed viruses), vaccine (including, but not limited to intranasally administered vaccines such as, for example, seasonal flu vaccines and coronavirus vaccines), and/or pharmaceutical agent (such as, for example zanamivir, oseltamivir, peramivir, baloxavir, midazolam, lorazepam, flumazenil, dexmedetomidine, ketamine, fentanyl, hydromorphone, butorphanol, naloxone, insulin, fluticasone, ciclesonide, budesonide, dupilumab, mometasone, albuterol, reslizumab, zileuton, mepolizumab, omalizumab, and haloperidol); wherein the GO nanoparticle is pegylated forming a GPP nanosheet.

The microbial antigen can be any viral, bacterial, and/or fungal antigen known in the art. For example, disclosed herein are compositions comprising i) a functionalized graphene oxide (GO) nanoparticle comprising polyethyleneimine (PEI) (GO-PEI, GP) and ii) a microbial antigen (such as for example, peptides, polypeptides, and proteins). For example, disclosed herein are compositions comprising a functionalized graphene oxide (GO) nanoparticle (including, but not limited to nanoparticle sheets) comprising polyethyleneimine (PEI) (GO-PEI, GP) and ii) a microbial antigen wherein the microbial antigen comprises a viral antigen from a virus selected from the group consisting of Herpes Simplex virus-1 (such as, for example, glycoprotein D and/or glycoprotein G), Herpes Simplex virus-2 (such as, for example, glycoprotein D and/or glycoprotein G), Varicella-Zoster virus (such as, for example, glycoprotein E), Epstein-Barr virus (such as, for example the EBV glycoprotein), Cytomegalovirus (such as, for example the CMV glycoprotein), Human Herpes virus-6, Variola virus, Vesicular stomatitis virus, Hepatitis A virus, Hepatitis B virus (including, but not limited to the hepatitis B virus surface antigen), Hepatitis C virus (such as, for example, the Hepatitis C E1, E2, or E3 proteins), Hepatitis D virus, Hepatitis E virus, Rhinovirus, Coronavirus (including, but not limited to spike or envelope proteins from avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A (such as, for example the hemagglutinin (HA) protein including the HA1 and HA2 protein and including trimeric HA), Influenza virus B (such as, for example the hemagglutinin (HA) protein including the HA1 and HA2 protein and including trimeric HA), Measles virus (such as, for example the hemagglutinin protein), Polyomavirus, Human Papilomavirus, Respiratory syncytial virus (such as, for example the RSV G protein), Adenovirus, Coxsackie virus, Dengue virus (such as, for example capsid protein, envelope protein, and/or premembrane/membrane protein), Mumps virus, Poliovirus, Rabies virus (including, but not limited to the Rabies glycoprotein), Rous sarcoma virus, Reovirus, Yellow fever virus, Zika virus (such as, for example capsid protein, envelope protein, and/or premembrane/membrane protein), Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A (including, but not limited to viral protein 4 and viral protein 7), Rotavirus B (including, but not limited to viral protein 4 and viral protein 7), Rotavirus C (including, but not limited to viral protein 4 and viral protein 7), Sindbis virus, Simian Immunodeficiency virus, Human T-cell Leukemia virus type-1, Hantavirus, Rubella virus, Simian Immunodeficiency virus, Human Immunodeficiency virus type-1 (such as, for example, glycoprotein (gp), envelope protein (Env), or gag protein), and Human Immunodeficiency virus type-2.

The disclosed functionalized GO-PEI nanoparticles (including, but not limited to nanoparticle sheets) are particularly useful in generating immune responses to respiratory illnesses such as influenza. Influenza virus is a member of Orthomyxoviridae family. There are three subtypes of influenza viruses, designated influenza A, influenza B, and influenza C. The influenza virus contains a segmented negative-sense RNA genome, which encodes the following proteins: hemagglutinin (HA), neuraminidase (NA), matrix (Ml), proton ion-channel protein (M2), nucleoprotein (NP), polymerase basic protein 1 (PB1), polymerase basic protein 2 (PB2), polymerase acidic protein (PA), and nonstructural protein 2 (NS2). The HA, NA, Ml, and M2 are membrane associated, whereas NP, PB1, PB2, PA, and NS2 are nucleocapsid associated proteins. The HA and NA proteins are envelope glycoproteins, responsible for virus attachment and penetration of the viral particles into the cell, and the sources of the major immunodominant epitopes for virus neutralization and protective immunity. Influenza A viruses are classified into subtypes based on antibody responses to HA and NA. These different types of HA and NA form the basis of the H and N distinctions in, for example, H5N1. There are 16 H and 9 N subtypes known, but only H 1, 2 and 3, and N 1 and 2 are commonly found in humans. Both HA and NA proteins are considered the most important components for prophylactic influenza vaccines. In one aspect, the microbial antigen can be from an Influenza A H1N1, H1N2, H2N2, H3N2, H5N1, H7N3, H7N7, H7N9, and/or H9N2 HA polypeptides for eliciting a broadly reactive immune response to H1N1, H1N2, H2N2, H3N2, H5N1, H7N3, H7N7, H7N9, and/or H9N2 influenza virus. Similarly, the influenza virus can be an influenza B virus including Victoria and Yamagata lineages.

Hemagglutinin (HA) is an influenza virus surface glycoprotein. HA mediates binding of the virus particle to a host cells and subsequent entry of the virus into the host cell. The nucleotide and amino acid sequences of numerous influenza HA proteins are known in the art and are publically available, such as those deposited with GenBank. HA (along with NA) is one of the two major influenza virus antigenic determinants. In one aspect, the microbial antigen can be HA.

As used herein, a matrix (Ml) protein refers to the influenza virus structural protein found within the viral envelope. Ml is thought to function in assembly and budding. As used herein, a neuraminidase (NA) refers to the influenza virus membrane glycoprotein. NA is involved in the destruction of the cellular receptor for the viral HA by cleaving terminal sialic acid residues from carbohydrate moieties on the surfaces of infected cells. NA also cleaves sialic acid residues from viral proteins, preventing aggregation of viruses. NA (along with HA) is one of the two major influenza virus antigenic determinants.

Despite the efficacy in treating, inhibiting, reducing, decreasing, ameliorating, and/or preventing viral infections, the disclosed compositions comprising i) a functionalized graphene oxide (GO) nanoparticle (including, but not limited to nanoparticle sheets) comprising polyethyleneimine (PEI) (GO-PEI, GP) and ii) a microbial antigen (such as for example, peptides, polypeptides, and proteins) are not limited to microbial antigens that are viral in origin, but can include bacterial antigens. Thus, disclosed herein are compositions comprising i) a functionalized graphene oxide (GO) nanoparticle comprising polyethyleneimine (PEI) (GO-PEI, GP) and ii) a microbial antigen, wherein the microbial antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Alternatively, or in addition, the microbial antigen can also be obtained from a fungal source. Accordingly, disclosed herein are compositions comprising i) a functionalized graphene oxide (GO) nanoparticle (including, but not limited to nanoparticle sheets) comprising polyethyleneimine (PEI) (GO-PEI, GP) and ii) a microbial antigen, wherein the microbial antigen comprises a fungal antigen from a fungi selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium mameffi, and Alternaria alternata.

It is understood and herein contemplated that achieving the proper ratio of GO-PEI particles to microbial antigen, vaccine, and/or pharmaceutical agent can go a long way to increasing the efficacy of the disclosed compositions in inducing microbial and/or vaccine specific immune responses and offering protective and/or therapeutic immune responses. In some aspects, disclosed herein are compositions comprising i) a functionalized graphene oxide (GO) nanoparticle comprising polyethyleneimine (PEI) (GO-PEI, GP) and ii) a microbial antigen, wherein the weight-to-weight ratio of GO-PEI particles to microbial antigen from 10:1 to 1:10, for example, 10:1, 8:1, 6:1, 5:1, 4:1, 3:1, 2:1, 1:1, 1:2, 1:4, 1:5, 1:6, 1:8, or 1:10.

While the combination of a GO-PEI nanoparticle and a microbial antigen, vaccine, and/or pharmaceutical agent are sufficient to offer increased antigen specific immune responses over the antigen alone and induce protective and/or therapeutic immune responses, it is understood and herein contemplated that the disclosed compositions can benefit from the addition of an adjuvant. As used herein, an “adjuvant” is any substance which enhances an immune response to an antigen. As defined, the PEI already has some adjuvant properties as does the GO-PEI nanoparticles. Nonetheless, other adjuvants can be incorporated into the GO-PEI and microbial antigen composition, including but not limited to CpG oligonucleotides (CpG ODN). Other suitable adjuvants include, but are not limited to AS04, MF59, AS01, CpG ODN1018, CpG ODN1826, tetanus toxoid, cholera toxin B subunit, diphtheria toxin CRM197, Adenylate cyclase toxoid mutant, pertussis toxin mutant, lipopolysaccharide (LPS), and/or Aluminum. As with the ratio of GO-DEI nanoparticles to microbial antigen particles is significant to the ultimate efficacy of the disclosed nanoparticles for inducing immune responses, so to is the ratio of GO-DEI to microbial antigen to adjuvant. Accordingly, disclosed herein are compositions comprising i) a functionalized graphene oxide (GO) nanoparticle comprising polyethyleneimine (PEI) (GO-PEI, GP) and ii) a microbial antigen, vaccine, and/or pharmaceutical agent, further comprising an adjuvant (such as, for example, CpG oligonucleotide (CpG ODN)) wherein the weight-to-weight ratio of GO-DEI to microbial antigen to adjuvant is 10:5:2.5 or 10:5:1.

Once formed, the disclosed particles have a diameter of less than 500 nm. In some instances the diameter of the GO-DEI nanoparticle and incorporated microbial antigen, vaccine, and/or pharmaceutical agent with or without an adjuvant is less than or equal to 200 nm, between 160-200 nm, between 160 and 170 nm, including but not limited to 150, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 215, 220, 225, 230, 235, 240, 245, 250, 255, 260, 265, 270, 275, 280, 285, 290, 295, 300, 310, 320, 330, 340, 350, 360, 370, 380, 390, 400, 425, 450, 475, 500 nm.

Once made, the nanoparticles have a tunable zeta potential from −35 mV to 35 mV. For example, the zeta potential can be −35, −34, −33, −32, −31, −30, −29, −28, −27, −26, −25, −24, −23, −22, −21, −20, −19, −18, −17, −16, −15, −14, −13, −12, −11, −10, −9, −8, −7, −6, −5, −4, −3, −2, −1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 mV. In one aspect, the zeta potential can be greater than 30 mV, for example, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.5, 34.0, 34.5 or 35 mV.

The disclosed compositions comprising compositions comprising i) a functionalized graphene oxide (GO) nanoparticle (including, but not limited to nanoparticle sheets) comprising polyethyleneimine (PEI) (GO-PEI, GP) and ii) a microbial antigen, vaccine, and/or pharmaceutical agent can be formulated as a vaccine for administration to a subject to inhibit or prevent infection upon future antigenic exposure. Thus, in one aspect, disclosed herein are vaccines comprising the compositions comprising i) a functionalized graphene oxide (GO) nanoparticle comprising polyethyleneimine (PEI) (GO-PEI, GP) and ii) a microbial antigen. Such vaccines can further include any adjuvant disclosed herein.

### 1. Homology/Identity

It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Nat. Acad. Sci. USA 86:7706-7710, 1989, Jaeger et al. Methods Enzymol. 183:281-306, 1989 which are herein incorporated by reference for at least material related to nucleic acid alignment.

### 2. Peptides

### a) Protein Variants

As discussed herein there are numerous variants of the influenza A HA1 protein, influenza A HA2 protein, influenza B HA1 protein and influenza B HA2 protein that are known and herein contemplated. In addition, to the known functional microbial antigen strain variants there are derivatives of the microbial antigen proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 3. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Methods of Making the Compositions

In one aspect, disclosed herein are methods of making a vaccine to a microbial antigen comprising a) obtaining a graphene oxide (GO) powder; b) sonicating GO flakes in an ice bath until the GO particles are less than 500 nm in diameter (such as for example, less than or equal to 200 nm, between 160-200 nm, between 160 and 170 nm, including but not limited to 150, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 215, 220, 225, 230, 235, 240, 245, 250, 255, 260, 265, 270, 275, 280, 285, 290, 295, 300, 310, 320, 330, 340, 350, 360, 370, 380, 390, 400, 425, 450, 475, 500 nm) using tip ultrasonication; c) adding polyethyleneimine (PEI) and sonicating; d) activating the GO-PEI with EDC; and e) adding the microbial antigen to the activated GO-PEI particles at a GO-PEI to Ag weight-to-weight ratio of 10:1, 8:1, 6:1, 5:1, 4:1, 3:1, 2:1, 1:1, 1:2, 1:4, 1:5, 1:6, 1:8, or 1:10.

Also disclosed herein are methods of making a vaccine to a microbial antigen, further comprising co-loading an adjuvant (such as, for example, CpG ODN) with the microbial antigen onto the GO-PEI nanoparticles. In some aspects, the weight-to-weight ratio of GO-PEI particles to microbial antigen to adjuvant comprises 10:5:2.5 or 10:5:1.

In some aspects, the GO-PEI nanoparticles and/or nanosheets can be further modified through PEGylation. Nanoparticle PEGylation can increase the stability and biocompatibility of the nanoparticles. Because of the charge shielding effect of PEG, PEGylation has become one of the most attractive strategies to improve the biocompatibility and physicochemical properties of diverse nanomedicines. PEGylated pulmonary surfactant-biomimetic nanoparticles have potentiated heterosubtypic influenza immunity of influenza vaccines and engineered mucus-penetrating drug carriers for sustained drug delivery at mucosal sites. The surface chemistry and storage stability of GPP nanosheets can be adjusted as needed by tuning the amount of conjugated PEG. The high versatility and flexibility of the GPP nanosheet scaffolds allow the co-incorporation of NP-M2e and hrHA successively to generate layered protein coatings, designed as hrHA/NP-M2e (outer layer/inner layer) Nano. Because of the GPP scaffold surface's high protein-loading capacity, the antigenic protein incorporation is nearly 100% efficient at scaffold to protein ratio from 1:2 to 2:1 at 4° C. overnight. We found that antigens in the outer shells of layered protein nanoparticles induced robust antibody responses while the antigens in the inner cores induced strong T cell responses. Therefore, we can generate new layered nanosheets by successive deposition of NP-M2e (as an inner layer) and hrHA (an outer layer) onto the new nanosheet scaffolds via electrostatic adsorption approach. A two-step process can prepare the nanosheet scaffolds. First, PEI will be conjugated to the carboxyl groups on GO via the Carbodiimide coupling method using N-(3-dimethyl aminopropyl-N′-ethyl carbodiimide) hydrochloride (EDC). Then, the GO-PEI nanosheets conjugate with PEG-NHS, designated as GPP nanosheets.

### D. Methods of Using the Compositions

It is understood and herein contemplated that the disclosed vaccines and compositions comprising i) a functionalized graphene oxide (GO) nanoparticle (including, but not limited to nanosheets) comprising polyethyleneimine (PEI) (GO-PEI, GP) and ii) a microbial antigen have significant benefit for delivering a microbial antigen to a subject and in particular being delivered to mucosal surfaces (including, but not limited to oral, rectal, vaginal, buccal, and/or respiratory surfaces). Thus, disclosed herein are methods of delivering an antigen to a mucosal surface of a subject comprising administering to a subject at risk of being infected with a microbe a vaccine as described herein or any of the compositions comprising i) a functionalized graphene oxide (GO) nanoparticle (including, but not limited to nanosheets) comprising polyethyleneimine (PEI) (GO-PEI, GP) and ii) a microbial antigen disclosed herein.

Once administered, the disclosed compositions comprising i) a functionalized graphene oxide (GO) nanoparticle comprising polyethyleneimine (PEI) (GO-PEI, GP) and ii) a microbial antigen and vaccines can elicit/induce immune responses specific to the microbial antigen in the composition and/or vaccine. Such responses can be mucosal immune responses like IgA antibody production, but can also include the generation of IgG antibodies, IL-6 production and TNF-α production. In one aspect, disclosed herein are methods of inducing an immune response (such as, for example, mucosal immune responses and/or production of microbial specific IgA antibodies, microbial specific IgG antibodies, IL-6 production, TNF-α production) in a subject to a microbial antigen comprising administering to the subject a vaccine as disclosed herein or any of the compositions comprising i) a functionalized graphene oxide (GO) nanoparticle comprising polyethyleneimine (PEI) (GO-PEI, GP) and ii) a microbial antigen described herein.

The induction of an immune response specific to the microbial antigen in the disclosed compositions and vaccines is particularly useful in the treatment of and/or immunization against an infection with the microbe from which the microbial antigen derives. Thus, disclosed herein are methods of treating, inhibiting, reducing, decreasing, ameliorating and/or preventing a microbial infection comprising administering to a subject at risk of being infected or that is infected with a microbe, any of the vaccines disclosed herein and/or any of the compositions comprising i) a functionalized graphene oxide (GO) nanoparticle (including, but not limited to nanosheets) comprising polyethyleneimine (PEI) (GO-PEI, GP) and ii) a microbial antigen described herein.

The infection can be a viral, bacterial, or fungal infection. In one aspect, the infection is a bacterial infection, wherein the infecting bacteria is selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

In some aspects, the infection is a viral infection of a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A (including, but not limited to H1N1, H1N2, H2N2, H3N2, H5N1, H7N3, H7N7, H7N9, and/or H9N2 influenza virus), Influenza virus B (including, but not limited to Victoria and Yamagata lineages), Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In some aspects, the infection is a fungal infection of a fungus selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis camii, Penicillium mameffi, and Altemaria altemata.

In some aspects, it is understood that some microbial antigens are highly conserved among strains of a microbe and administration of a particular microbial antigen can have cross-reactivity and provide a protective and/or therapeutic immune response to other strains of the same microbe. For example, use of a GO-DEI and an influenza A HA protein, from an H1N1 Influenza A virus can have a protective effect against infection with H1N2, H2N2, H3N2, H5N1, H7N3, H7N7, H7N9, and/or H9N2 in addition to the expected protection against H1N1. Similarly, immunization with a GO-DEI nanoparticle and a coronavirus spike protein can have a protective and/or therapeutic immune response against any other coronavirus including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV.

### E. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Intranasal Vaccination with Influenza HA/GO-PEI Nanoparticles Provides Broad Immune Protection Against Homo- and Heterologous Strains

Influenza remains one of the leading infectious diseases causing morbidity and mortality worldwide. Vaccination is the most cost-effective approach to preventing influenza virus infection. However, current virus-based seasonal influenza vaccines induce strain-specific immunity and are less effective against mismatched strains that may cause influenza epidemics. Furthermore, there is no vaccine countermeasure available for new pandemic strains. Intranasal (i.n.) immunization is a promising vaccination route for infectious respiratory diseases, such as influenza. This vaccination route can induce both systemic and mucosal immune responses. Secretory immunoglobulin A (sIgA) and immunoglobulin G (IgG) may prevent influenza infection at the portal of virus entry. Influenza mucosal immunity has been reported to confer cross-protection against heterologous and heterosubtypic viruses. Moreover, needle-free intranasal influenza vaccines possess superior logistical advantages over traditional injectable vaccines, such as easy administration with high acceptance for recipients and avoidance of biohazardous sharps waste.

However, the development of i.n. influenza vaccines has progressed slowly. The cold-adapted live-attenuated influenza virus (LAIV) vaccine is the only available human i.n. influenza vaccine up to date. Studies have shown that the LAIV vaccine could provide heterologous immunity. Nevertheless, safety concerns of LAIV are raised, especially in high-risk populations, such as infants under 2-year-old and the elderly over 50. LAIV could undergo genetic reassortments and revert into a virulent form, thus posing a risk. Besides, the suboptimal protective efficacy of LAIV vaccines was reported in children in the 2009 H1N1 pandemic. A new generation of virus-independent, safe, and efficient influenza intranasal vaccines that induce broader cross-protection with high efficacy is urgently needed.

Intranasal vaccination with recombinant protein/peptide-based vaccines is an attractive strategy with high safety. Purified protein/peptide antigens eliminate safety concerns in individuals with egg allergies, possess minimal side effects, and could be produced quickly and cost-effectively. However, soluble protein vaccines are poorly immunogenic by i.n. immunization due to the harsh and tolerogenic mucosal environment. The selection of appropriate formulations and adjuvants is crucial for successful i.n. vaccines. Nanoparticle vaccine platforms have been applied for i.n. vaccine development in recent years. Nanoparticles serve as antigen and adjuvant carriers and immunostimulants themselves to enhance immune responses. The immunoenhancing effects of various nanoparticles have been reported. However, most nanoparticle vaccines suffer from low antigen-loading capacity, complicated and lengthy preparation procedures, and structural complexity because of covalent conjugation.

Two-dimensional (2-D) graphene oxide (GO) nanoparticles a great vaccine platform due to their extraordinary attributes. These features include the high aspect ratio and ultra-large surface area for high-density antigen association, wealthy chemical groups for flexible surface modification, and noncovalent antigen loading via electrostatic adsorption, hydrogen bond, and hydrophobic and π-π stacking interactions. Besides, GO nanoparticles themselves are biocompatible and nonimmunogenic. Various GO vaccine formulations were demonstrated to induce improved immune responses by activating immune cells or triggering innate signaling. However, most prior studies were limited to conventional routes with tumor antigens for cancer immunotherapies. Studies on GO-based influenza i.n. vaccines are lacking.

We developed a polyethyleneimine (PEI)-functionalized GO (GO-PEI, GP) influenza vaccine nanoplatform, prepared influenza GP nanoparticles by incorporating recombinant influenza HA, and investigated their immunoenhancing effects (FIG. 1). The work herein revealed that influenza GP nanoparticles enhanced antigen internalization and promoted the production of inflammatory cytokines and the maturation of JAWS II dendritic cells (DCs) during in vitro experiments. Intranasal vaccination with influenza GP nanoparticles induced robust humoral and cellular immune responses, conferring broader protection against both homologous and heterologous virus challenges in mice.

### a) Results

### (1) Fabrication and Characterization of GO Nanoparticle Vaccines

GO nanoparticle vaccines can be prepared in several approaches. We found that simple mixtures of naked GO and proteins are prone to precipitation and the protein loading capacity is low by direct surface adsorption. One of the best ways to engineer GO-based vaccine delivery systems is surface functionalization, which tailors the interactions between GO nanoparticles, vaccine components, and biosurfaces, and adjusts the adjuvant activity. We functionalized GO with a cationic polymer, branched PEI, to construct GO-PEI (GP) nanoparticles (FIG. 1A). Positively-charged nanoparticles are especially suitable for mucosal vaccination. Recent studies also showed that PEI has a potent mucosal adjuvant effect.

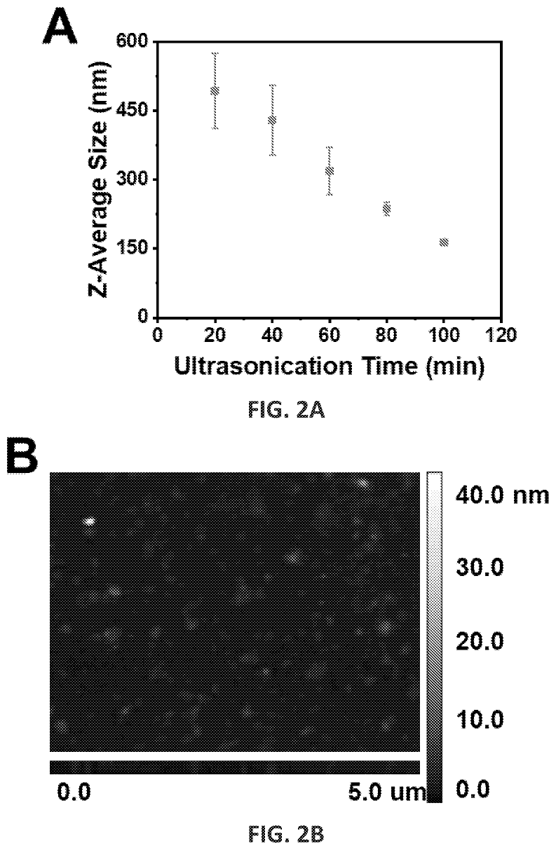

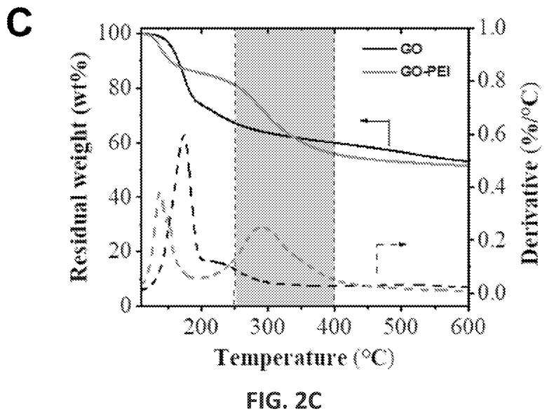

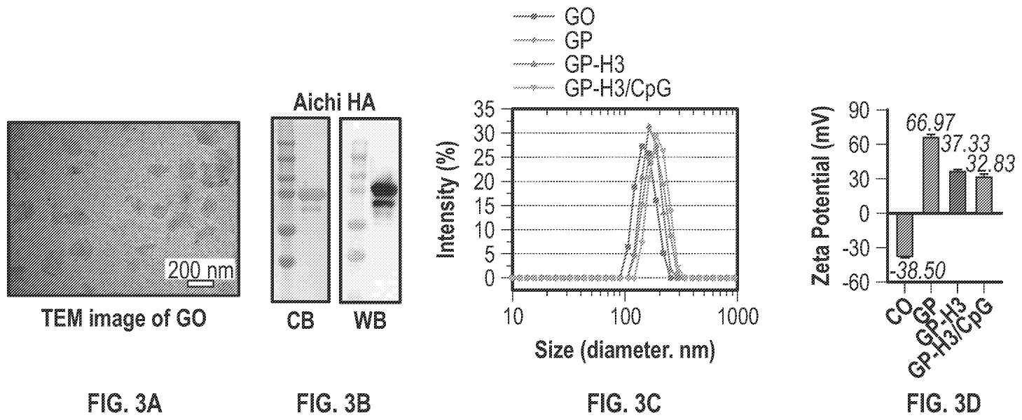

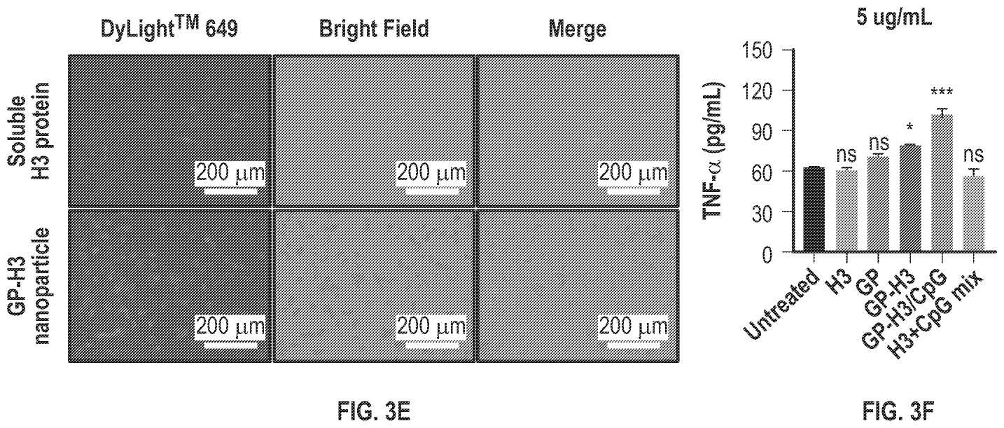

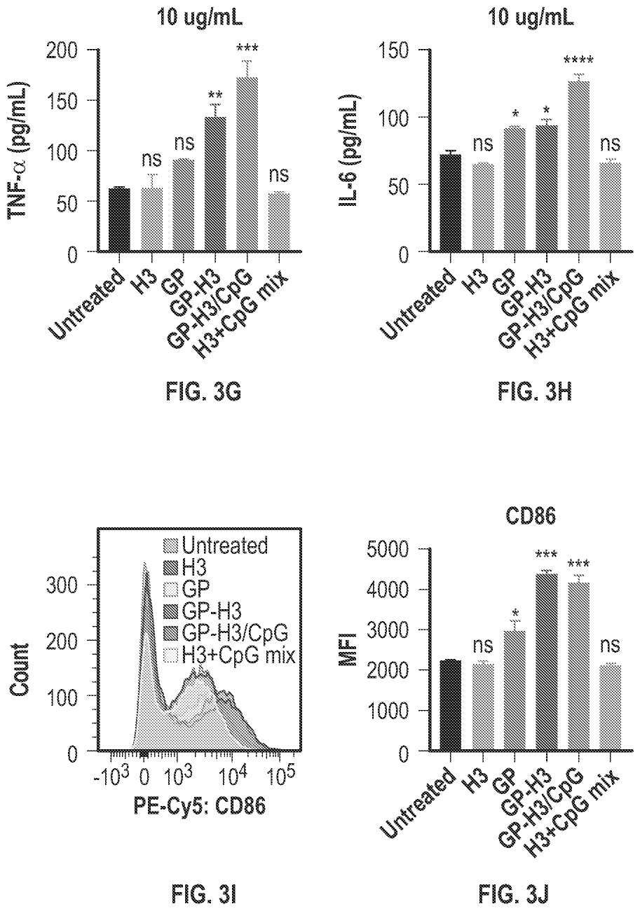

The pristine GO nanoparticles were prepared by tip sonication of GO flakes in an ice bath. The GO flakes gradually became smaller nano-sized GO nanoparticles upon sonication (FIG. 2A), and the final nanoparticles were around 164 nm. Transmission electron microscopy (TEM) and atomic force microscopy (AFM) images revealed the sheet-like (2-D) morphology (i.e., nanosheet) and uniform size distribution of the GO nanoparticles (FIG. 3A and FIG. 2B). We prepared PEI-functionalized GO (GO-PEI, GP) nanoparticles using the Carbodiimide coupling method. The thermo-gravimetric analysis (TGA) indicated that 17.94% PEI was conjugated on the GP nanoparticles (FIG. 2C). The resulting GO-PEI nanoparticle was 185±1.94 nm (FIG. 3C). From pristine GO to positive GO-PEI, the surface charge changed from negative (−38.5±0.51 mV) to positive (66.97±1.65 mV) (FIG. 3D), facilitating a high loading capacity of the protein antigens by direct electrostatic adsorption without chemical conjugation. Notably, compared with GO nanoparticles, GP has improved dispersibility and stability in saline solutions. PEI-functionalized GO was reported to be biocompatible.

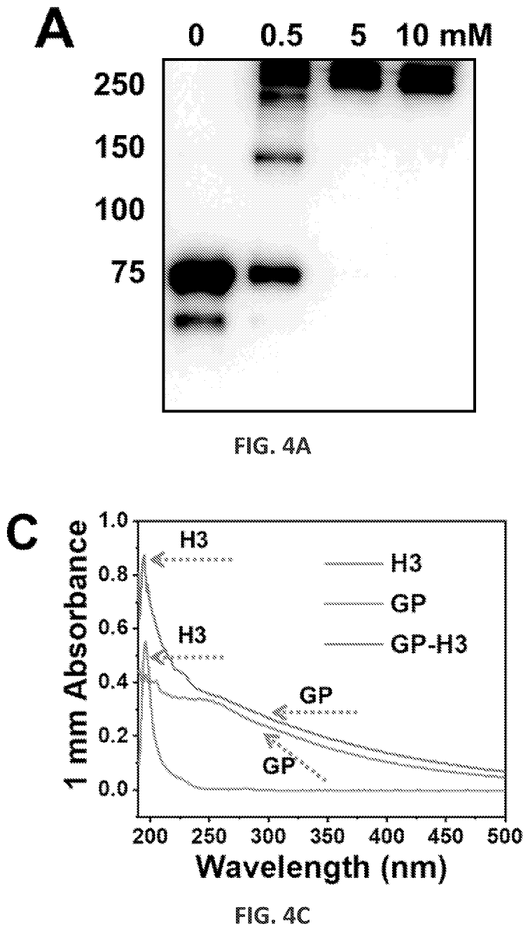

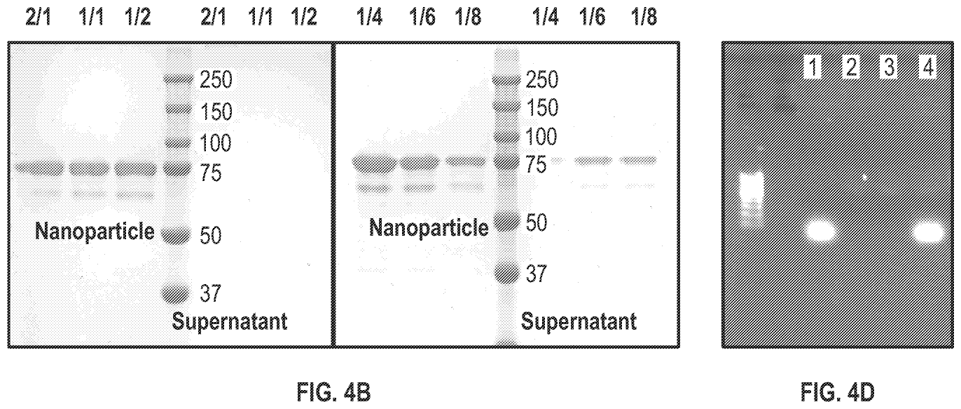

We have demonstrated that GCN4-stabilized trimetric influenza HA possesses enhanced immunogenicity compared with monomeric HA. We purified recombinant trimeric Aichi HA (A/Aichi/2/1968 (Aic), H3N2, designated H3) with high purity, as determined by SDS-PAGE followed by Coomassie Blue staining (FIG. 3B). The retention of HA antigenicity was verified by Western blotting analysis with antibodies. The trimeric state of H3 was determined by bis[sulfosuccinimidyl] suberate (BS3) crosslinking, followed by Western blots (FIG. 4A). A main band with a three-fold molecular weight of the monomeric HA was seen after a high concentration of BS3 (>5 mM) treatment to fix the polymeric HA, indicating the dominant trimeric state of the purified H3.

We generated GP nanoparticle vaccine formulations by a simple mixing/adsorption approach, as diagrammed in FIG. 1A. H3 was bound onto the large surface of GP nanoparticles mainly via electrostatic interactions. The antigen-loading capability of GP nanoparticles was evaluated by SDS-PAGE. At higher ratios of GP to H3 (2:1, 1:1, and 1:2), there were strong H3 bands from the nanoparticles without visible signals from the supernatants (FIG. 4B), indicating the tight binding of H3 onto the GP nanoparticles. Some free H3 was present in the supernatants from lower GP:H3 ratios, indicating an excessive amount of H3 in this circumstance. The resulting GP-H3 nanoparticles had strong colloidal stability, especially at higher GP:H3 ratios. UV-Vis absorption spectra analysis in FIG. 4C further confirmed the tight binding of H3 on GP nanoparticles. The results demonstrated that GP nanoparticles with positive charges and ultra-large surface areas can serve as protein antigen carriers with high antigen-loading capacities.

CpG ODN is a potent mucosal immunomodulator for the induction of antigen-specific cell-mediated immunity and humoral immune responses. We used CpG ODN1826 as a positive control group in this work. Meanwhile, negatively charged CpG molecules can be easily co-loaded with influenza HA onto the GP particles to generate self-adjuvanted nanoparticles. Agarose gel electrophoresis was employed to investigate whether all the feeding CpG were complexed and loaded onto the GP nanoparticles. As shown in FIG. 4D, strong CpG signals were observed from free CpG and H3+CpG but not from the supernatants of GP-H3/CpG (10:5:1) and GP-H3/CpG (10:5:2.5). The results indicated that all the feeding CpG was co-loaded with H3 on the GO-PEI nanoparticles via electrostatic adsorption interaction. Additionally, the obtained GP-H3/CpG (10:5:1) nanoparticles displayed a Zeta-potential value of 32.83±0.49 mV (FIG. 3D), which indicated that the resulting nanoparticles retained positive charges after loading with H3 and CpG. For subsequent studies, a GP:H3:CpG ratio of 10:5:1 was used for fabricating GP vaccine nanoparticles. The resulting GP-H3 and GP-H3/CpG nanoparticles ranged from 170 to 200 nm in diameters (FIG. 3C) and exhibited Zeta potentials of >+30 mV (FIG. 3D). The results demonstrated that GP-based H3 vaccine nanoparticles can be generated by the facile mixing method with appreciable particle features for i.n. immunization.

### (2) Effects on Murine Dendritic Cell Line JAWS II

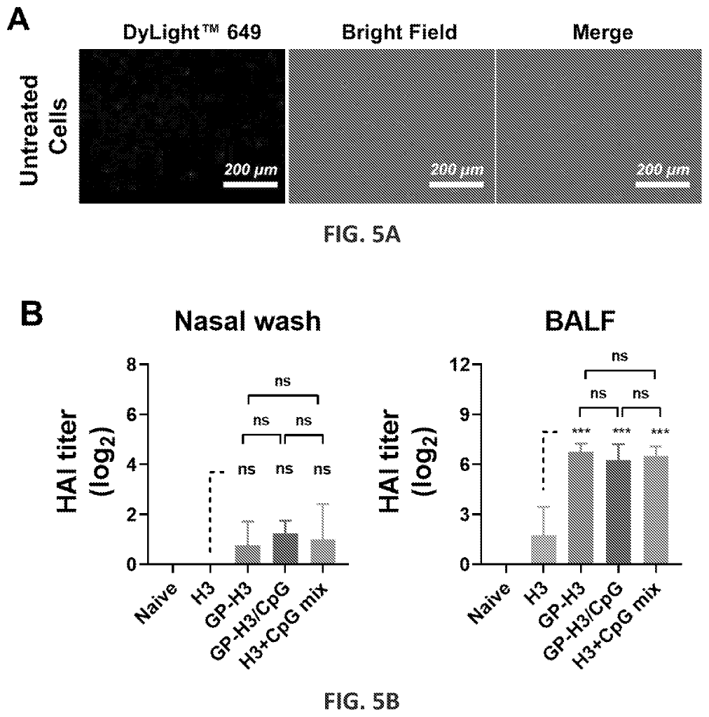

Effective antigen uptake by DCs is critical for inducing potent immune responses. We detected H3 internalization by immunofluorescence imaging in JAWS II cells treated with soluble H3 vs. GP-H3 nanoparticles. The weak background fluorescence in the untreated cell controls indicated low nonspecific adsorption of fluorescent antibodies (FIG. 5A). Soluble H3-treated JAWS II cells showed a weak fluorescence, indicating the low internalization of the H3 protein (FIG. 3E). In comparison, GP-H3 nanoparticle-treated cells displayed strong fluorescence, demonstrating significantly enhanced antigen uptake efficacy in DCs. Besides, the immunofluorescence imaging results also confirmed that the specificity and function of H3 on GP-H3 nanoparticles were well maintained after loading on GP. These results revealed that GP-based HA nanoparticles were readily taken in by dendritic cells.

Mature DCs produce cytokines to facilitate the activation and differentiation of T cells and regulate adaptive immunity. We evaluated the secreted cytokine (IL-6 and TNF-α) levels from JAWS II cells treated with different vaccine formulations. As shown in FIGS. 3F and 3G, soluble H3, GP, or H3+CpG induced comparable TNF-α secretion levels to the control, while GP-H3 and GP-H3/CpG nanoparticles induced significantly higher levels of TNF-α secretion at both H3 concentrations. Meanwhile, soluble H3 and H3+CpG groups showed similar IL-6 production levels to the control group in JAWS II cells (FIG. 3H). In comparison, GP or GP-H3 treatment significantly enhanced IL-6 production (P<0.05). The low TNF-α and IL-6 secretion in the H3+CpG group could indicate the limited internalization of the soluble protein and CpG into JAWS II cells. Although soluble CpG did not influence the cytokine production, CpG-loaded GP-H3/CpG nanoparticles showed a substantial enhancement over GP-H3 nanoparticles. Among all groups, GP-H3/CpG treatment induced the highest cytokine production in JAWS II cells. Therefore, GP vaccine nanoparticles enormously boosted the production of proinflammatory cytokines in JAWS II cells.

We studied the maturation efficacy of JAWS II cells by evaluating a DC maturation marker CD86 with flow cytometry. Consistent with the cytokine secretion results, soluble H3 or H3+CpG-treated JAWS II cells showed background levels of CD86 expression (FIGS. 3I and 3J). In contrast, GP-H3 and GP-H3/CpG treatments showed significantly higher CD86 expression (P<0.001), as revealed by the red-shift of the fluorescence spectra and the enhanced mean fluorescence intensity. Interestingly, naked GP nanoparticle treatment enhanced the CD86 expression (P<0.05), demonstrating GP nanoparticles' adjuvant effect. Overall, these results showed that GP nanoparticles can be readily internalized by dendritic cells and promote the production of proinflammatory cytokines, IL-6 and TNF-α, and stimulate DC maturation.

### (3) Induction of Humoral Immune Responses

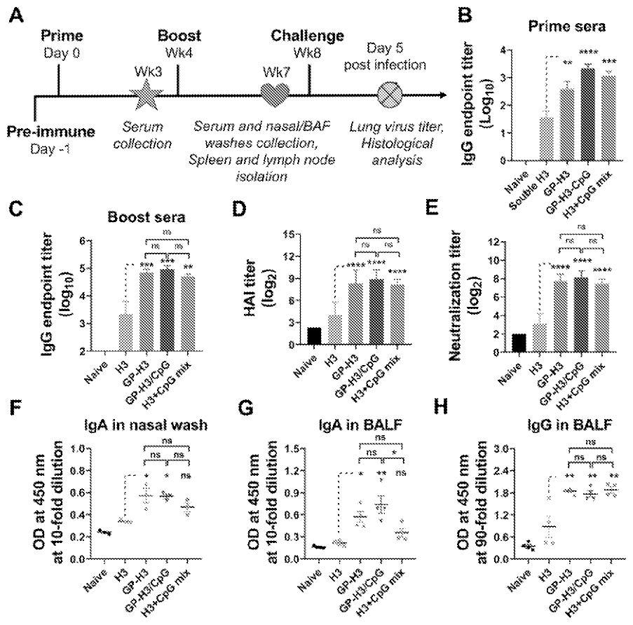

We investigated the GP-H3 nanoparticle immunogenicity by a two-dose vaccination scheme (FIG. 6A). Immunization groups include soluble H3, GP-H3, GP-H3/CpG and H3+CpG (5 μg of H3 per mouse). A previous study showed that a single intranasal dose of 10 or 20 μg of PEI was similarly safe as cholera holotoxin, CTB, and poly(lactic-co-glycolic acid) nanoparticles used at, or above, standard doses. In the present study, the PEI amount on GP nanoparticles was 1.79 μg per mouse (10 μg GP per mouse) determined by the TGA results.

Serum antigen-specific IgG levels were titrated (FIGS. 6B and 6C). The results demonstrated that the soluble H3 group displayed a low seroconversion efficiency and a low serum IgG level after the immunization. In contrast, GP-H3 nanoparticle immunization induced rapid seroconversion and significantly higher antigen-specific IgG antibody titers in both prime sera (p<0.01) and boost sera (p<0.001). H3+CpG significantly promoted the serum IgG responses compare to soluble H3. CpG has been proved in trials to accelerate the induction and generation of higher protective antibody titers with protein vaccines. All the GP nanoparticle (GP-H3 and GP-H3/CpG)-vaccinated mice showed high serum antigen-specific IgG titers after the boosting immunization. However, no significant difference was observed between GP-H3, GP-H3/CpG, and H3+CpG in antibody production.

Hemagglutination-inhibition (HAI) titer correlates to HA-induced immune protection. A serum HAI titer ≥40 is considered protective. As shown in FIG. 6D, soluble H3-immunized mice displayed relatively low HAI titers. In contrast, GP-H3 nanoparticles induced significantly higher HAI titer (p<0.0001) compared to the H3 group. Although GP-H3/CpG group showed elevated HAI titers compared to GP-H3 and H3+CpG groups, the difference is not significant.

Antibodies can be neutralizing or non-neutralizing. Neutralizing antibodies inhibit viral infectivity by tightly binding to important viral structures and correlate immune protection for many vaccines. As shown in FIG. 6E, significantly higher Aichi virus-specific neutralization antibody titers were induced in GP-H3 (p<0.0001), GP-H3/CpG (p<0.0001) and H3+CpG mix (p<0.0001) groups compared to the soluble H3 group. H3 in GP nanoparticles induced comparable neutralizing activity to H3+CpG mix (p>0.05), and CpG added no advantage when co-incorporated into GP nanoparticles with H3 (GP-H3/CpG vs. GP-H3, p>0.05). The antibody neutralization result was consistent with the antibody and HAI titer results.

Intranasal vaccination can induce sIgA and IgG in the respiratory tract surfaces, preventing influenza infection at the viral entry site. The cross-reactive sIgA provided broad protection against heterologous and heterosubtypic influenza viruses. As shown in FIGS. 6F and 6G, soluble H3 induced low levels of sIgA in nasal washes and BALF. By contrast, GP-H3 and GP-H3/CpG nanoparticle groups displayed elevated IgA antibody levels, which are also higher than that of the H3+CpG mix group.

We also observed high IgG levels in BALF (FIG. 6H) but low IgG antibody levels in nasal washes. These results were consistent with observations that sIgA dominated the upper respiratory tract antibody response, whereas IgG was the major antibody isotype in the lower respiratory tracts. We observed that GP-H3 and GP-H3/CpG nanoparticles induced significantly higher IgG levels (p<0.01, FIG. 6H) and HAI titers (p<0.001, FIG. 5B) in BALF than soluble H3, comparable to the H3+CpG mix group, but HAI titers were not detected in nasal washes from all groups (FIG. 5B).

These results demonstrated that GP nanoparticles potently boosted antibody immune responses not only in systemic compartments but also at local mucosal surfaces. Co-loading CpG to GP nanoparticles with H3 did not further significantly strengthen antigen-specific antibody responses, indicating that GP nanoparticle is a robust adjuvant system itself and can mask the adjuvant effect of CpG.

### (4) Induction of Cellular Immune Responses

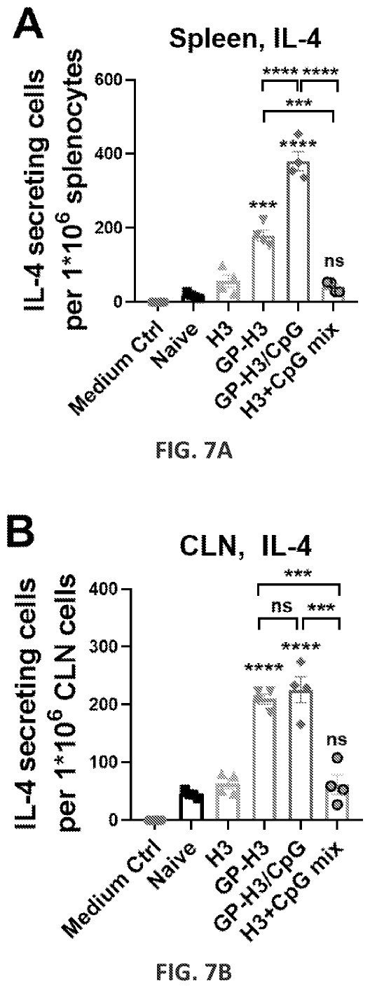

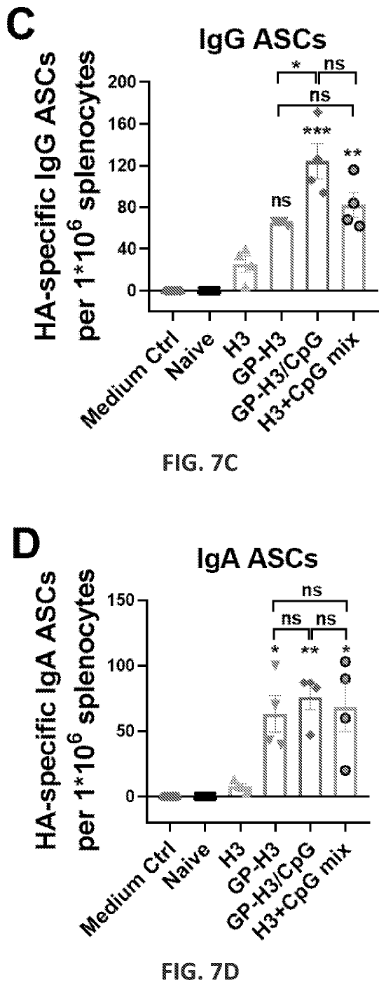

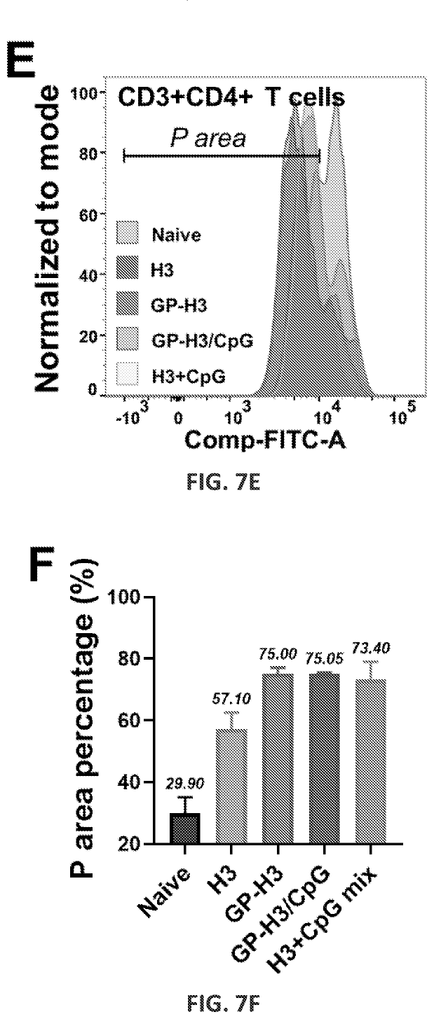

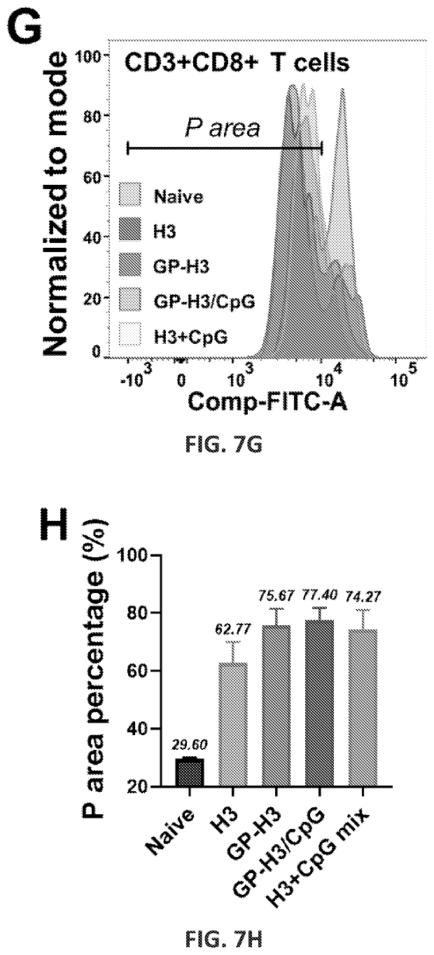

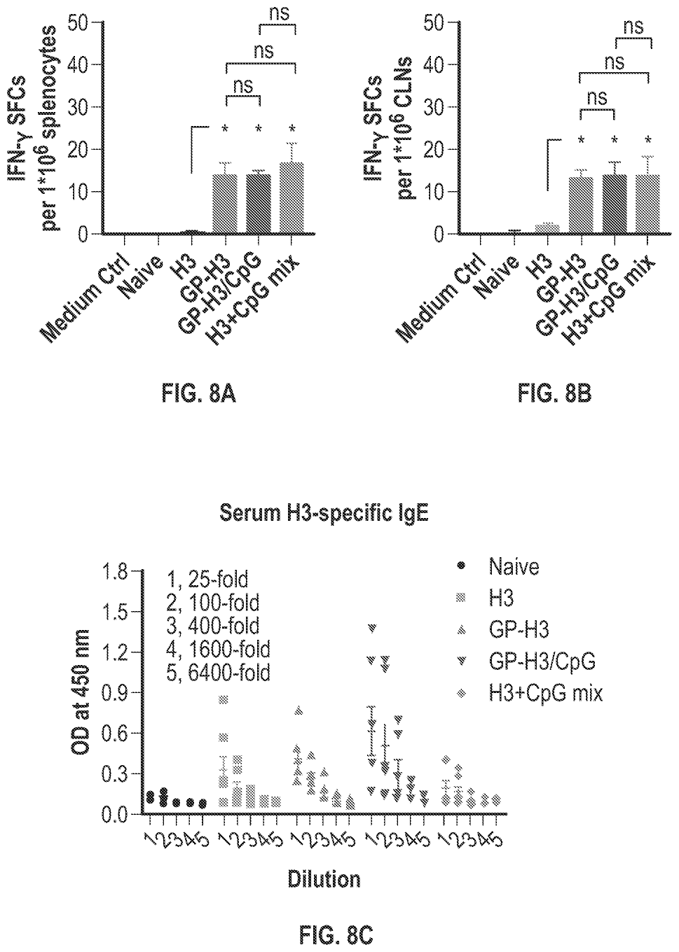

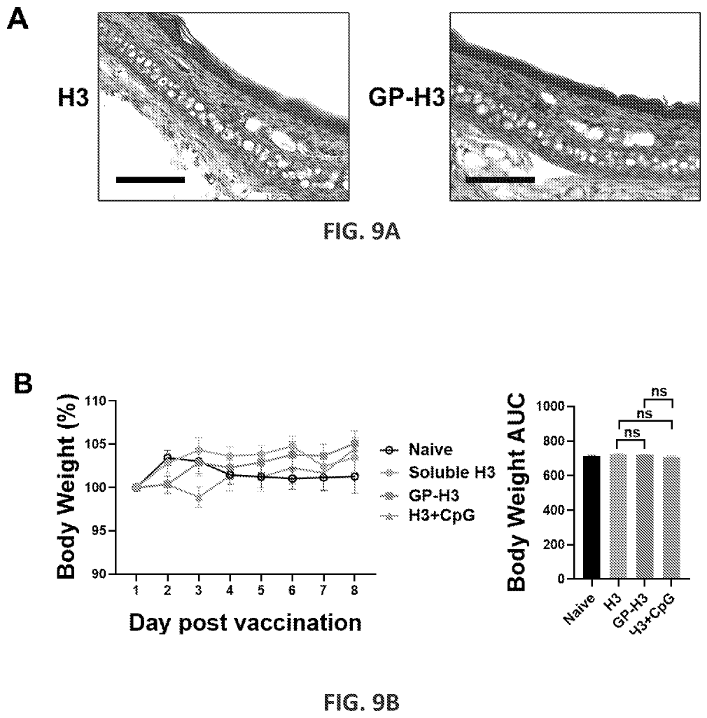

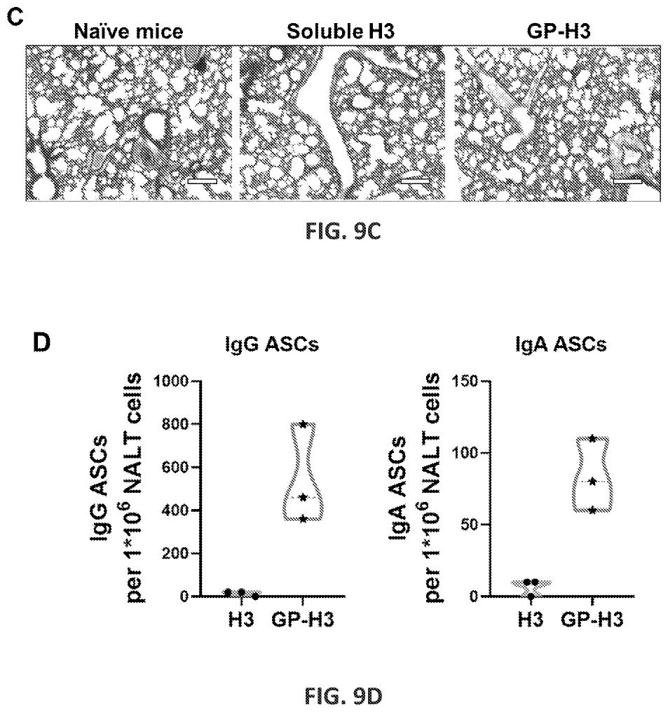

Cellular immunity plays a crucial role in the battle against influenza infection. Early cytokine production in vaccination programs the dimension and magnitude of antigen-specific immune responses. We evaluated IL-4-secreting cell frequencies in spleen and cervical lymph node (CLN) lymphocytes 3 wks after boosting immunization. As shown in FIG. 7A, we observed significantly increased numbers of IL-4-secreting splenocytes in the GP-H3 (p<0.001) and GP-H3/CpG (p<0.0001) nanoparticle groups vs. the soluble H3 or H3+CpG mix groups. We found no significant difference between soluble H3 and H3+CpG groups in the number of IL-4-secreting splenocytes (p>0.05). Similarly, GP nanoparticles boosted the IL-4-secreting lymphocyte generation in CLNs of vaccinated mice (FIG. 7B). Meanwhile, IFN-γ-producing lymphocytes were also observed in spleens and CLNs of nanoparticle-immunized mice (FIGS. 8A and 8B). GP-H3 nanoparticle immunization induced significantly more IFN-γ-producing lymphocytes than soluble H3. Considering the abundant IL-4 secreting cells observed, we measured the IgE antibody levels in boost sera. GP-H3 nanoparticle-vaccinated mice showed similar low IgE levels to the soluble H3 group (FIG. 8C). Besides, no visible inflammation was observed in the mouse nasal cavity 24 hours post i.n. immunization with GP-H3 nanoparticles (FIG. 9A). We also did not find apparent mouse body weight changes and inflammatory cell infiltration in lungs 7 days post-immunization (FIGS. 9B and 9C).

IL-4 facilitates B cell proliferation and differentiation into antibody-secreting plasma cells (ASCs). We studied the antigen-specific IgG and IgA ASCs in mouse spleens and analyzed whether IL-4-secreting lymphocyte frequency correlates with the antibody induction. As shown in FIGS. 7C and 7D, compared with soluble H3, GP-H3, and GP-H3/CpG nanoparticles induced increased numbers of H3-specific IgG and IgA ASCs in splenocytes. We also observed a similar plasma B cell pattern in nasal-associated lymphoid tissues (NALTs) (FIG. 9D). Therefore, GP nanoparticles significantly boosted antigen-specific plasma B cell generation.

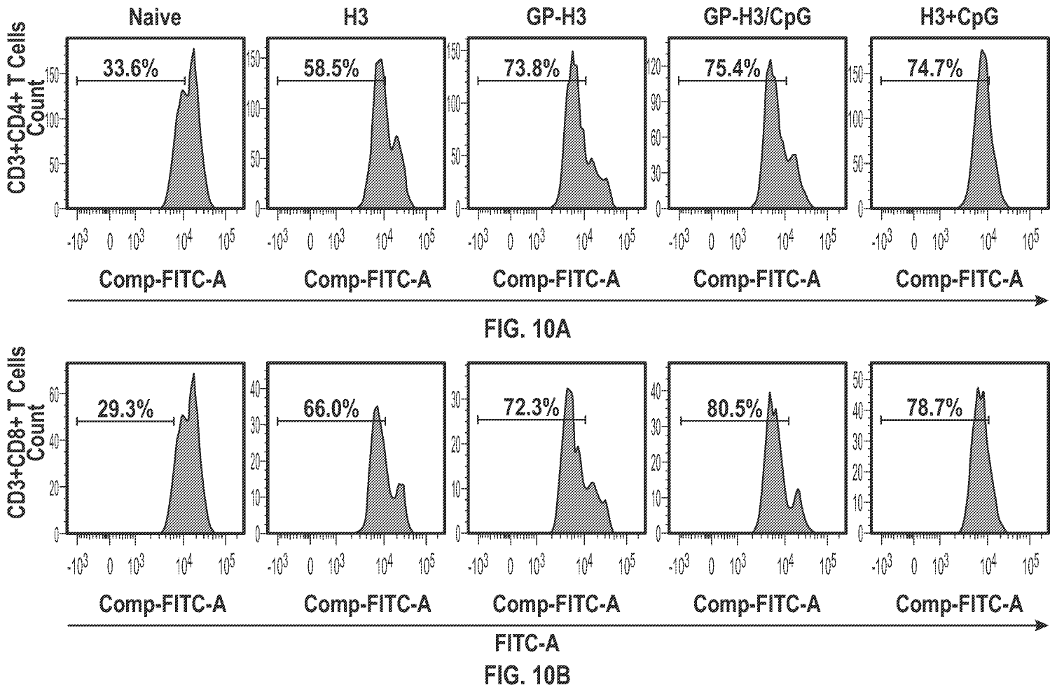

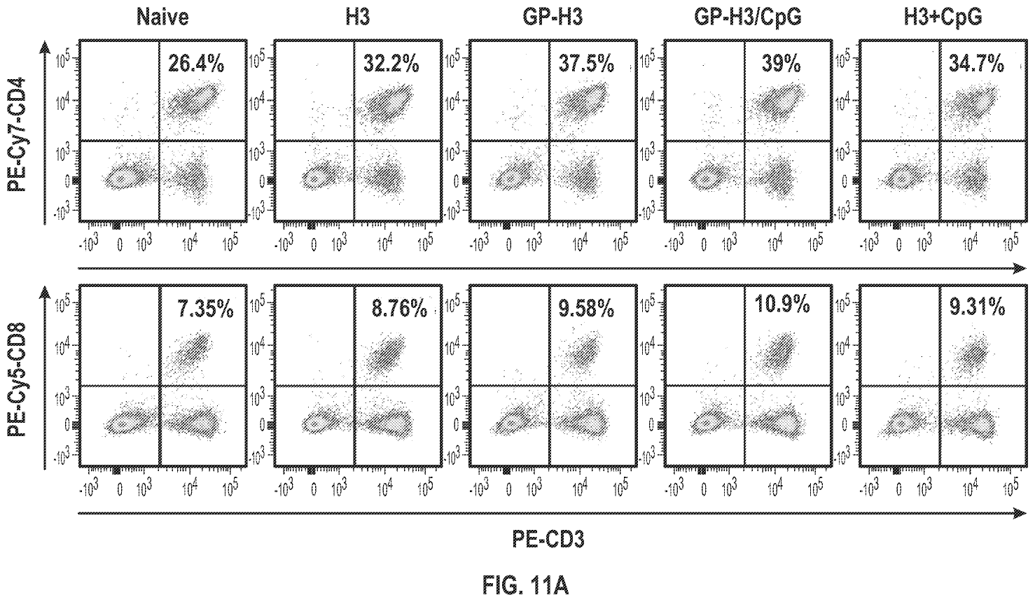

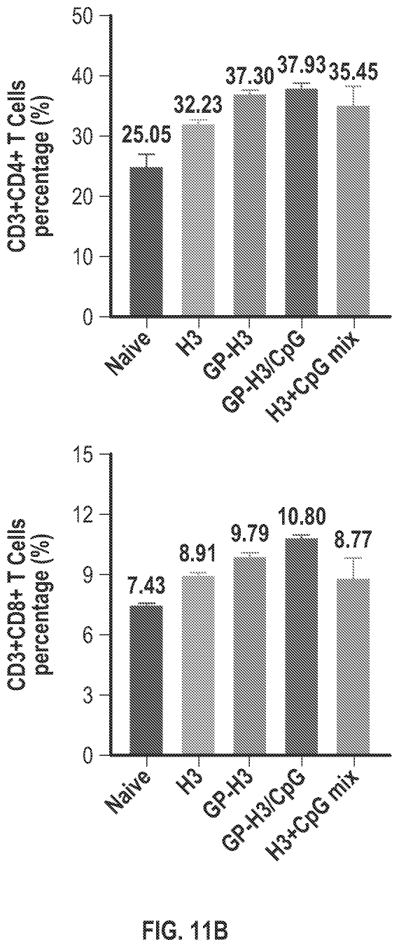

We evaluated CD4+ and CD8+ T cell proliferation in splenocytes by the CFSE staining assay (FIGS. 7E, 7F, 7G, and 7H, and FIG. 10). While all immunization groups showed a noticeable decrease in cellular CFSE fluorescence intensity over controls, higher CD4+ and CD8+ T cell proliferation was elicited in GP-H3 and GP-H3/CpG groups vs. the soluble H3 group. We also analyzed CD4+ and CD8+ T cell percentages in splenocytes after antigen re-stimulation (FIG. 11). Both GP-H3 and GP-H3/CpG groups displayed higher CD4+ and CD8+ T cell rates than the soluble H3 group in the bulk splenocytes. Therefore, GP nanoparticle vaccination significantly promoted cellular immune response, indicating the appreciable adjuvanticity of GP nanoparticles. Although CpG improved T cell responses in the context of H3+CpG mix, we did not observe the adjuvant effect in GP-H3/CpG nanoparticles (FIGS. 7E, 7F, 7G, and 7H).

### (5) Protective Efficacy Against Homologous Influenza Virus Challenge

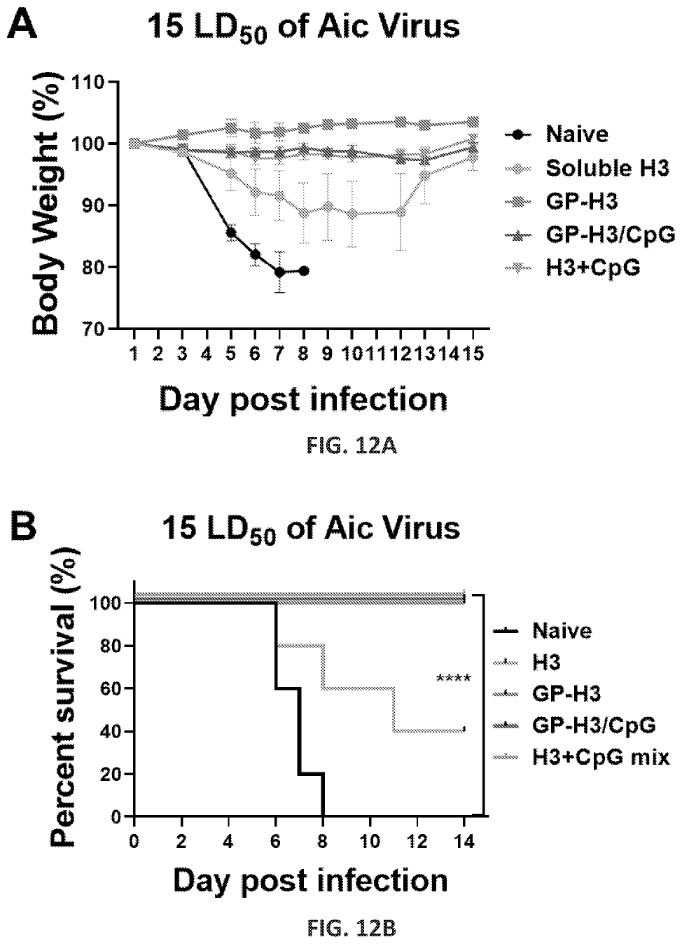

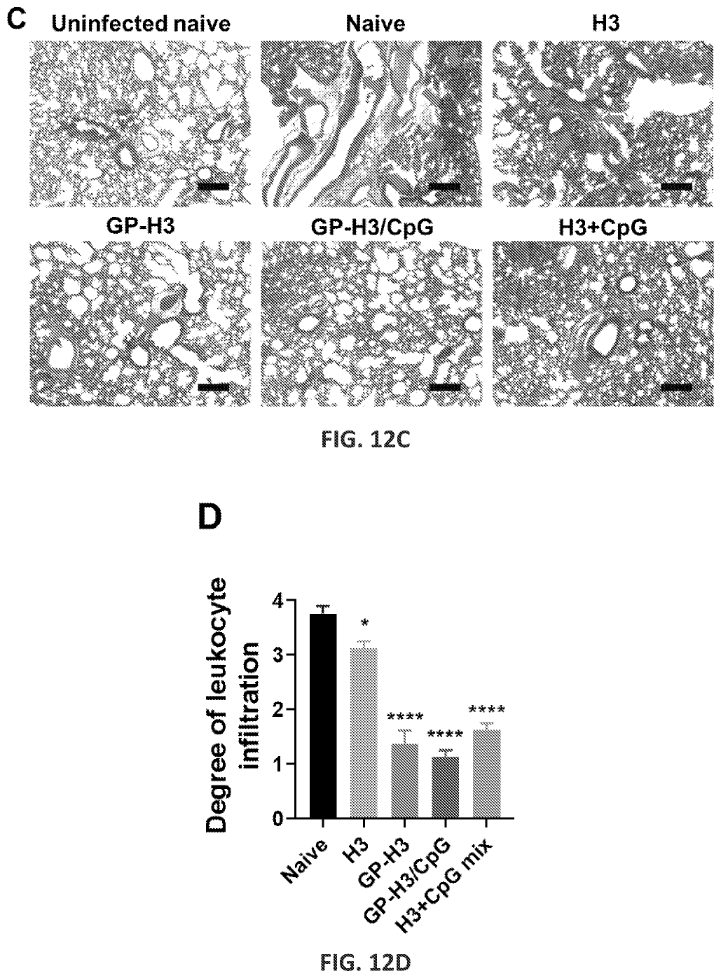

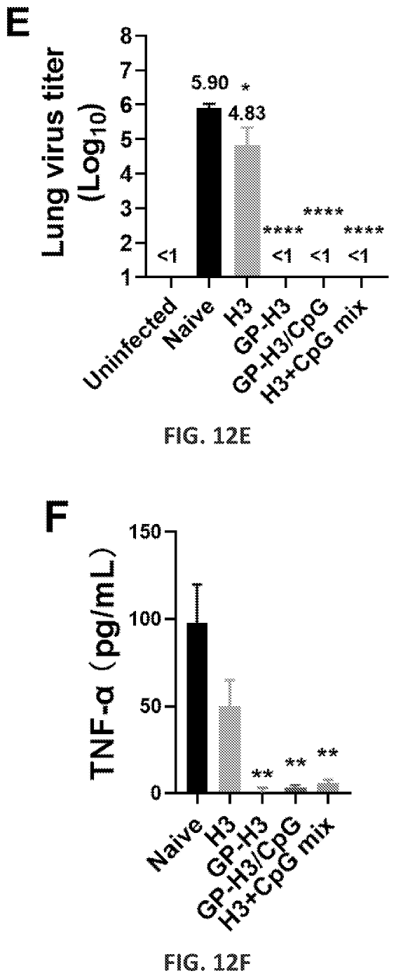

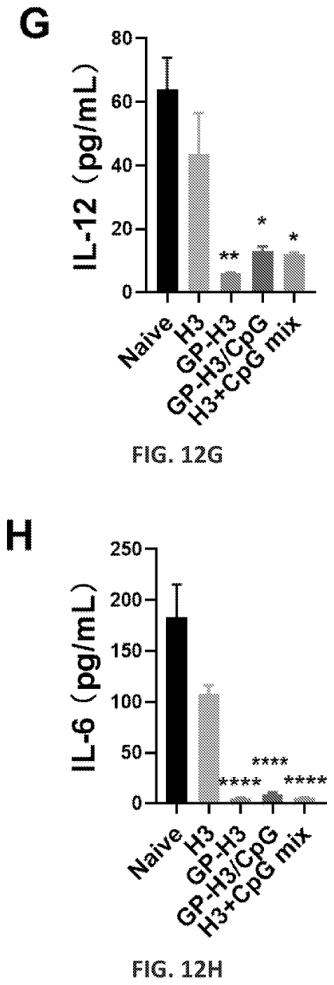

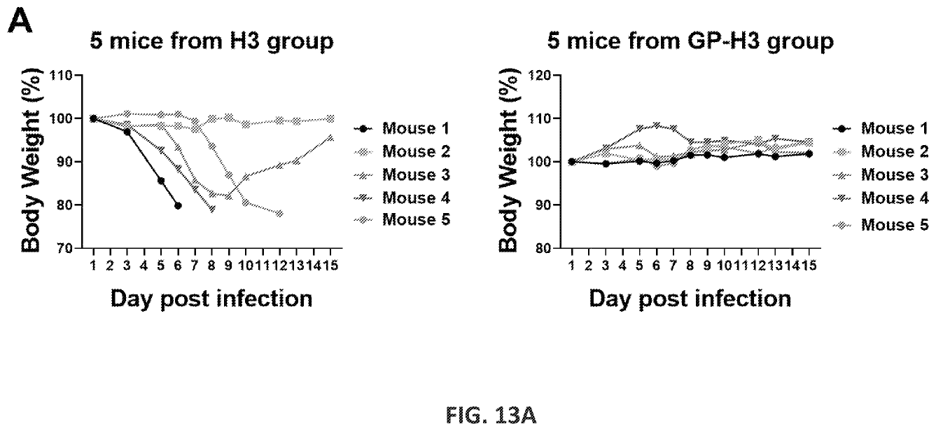

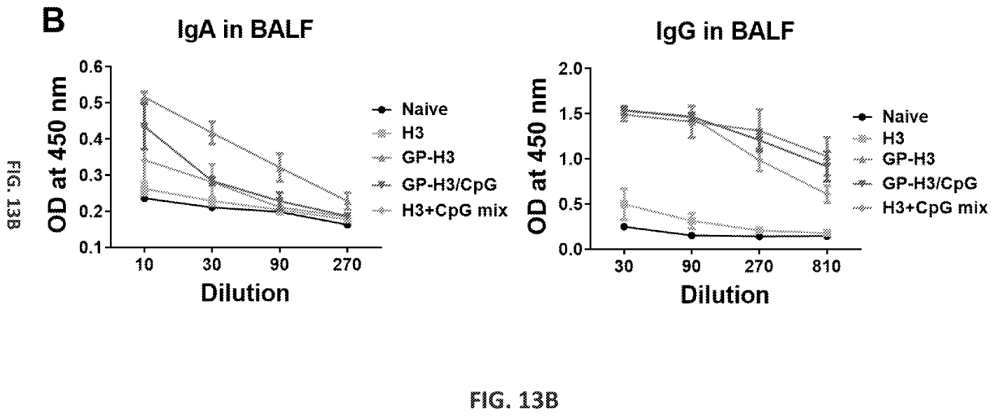

We investigated the prophylaxis efficiency of different formulations by challenging immunized mice with 15×LD₅₀ Aichi (FIGS. 12A and 12B). The soluble H3 immunization conferred partial protection (40% mouse survival) with apparent body weight loss (FIG. 13A). By contrast, mice in GP-H3, GP-H3/CpG, or H3+CpG mix immunization groups survived the challenge without significant weight loss. These results demonstrated that GP nanoparticle vaccines displayed superior protective effects vs. soluble H3.

We performed histological examinations and determined lung virus titers 5 days post-challenge. Naïve and soluble H3-immunized mice demonstrated a severe inflammatory state with massive tissue damage and leukocyte infiltration (FIG. 12C). By contrast, GP nanoparticle (GP-H3 and GP-H3/CpG) and H3-CpG mix-immunized mice displayed a nearly normal state with significantly decreased leukocyte infiltration (FIGS. 12C and 12D). Additionally, H3-immunized mice also displayed high lung virus titers (1×10^{4.83} TCID₅₀). In contrast, GP-H3, GP-H3/CpG, and H3-CpG mix groups showed undetectable lung virus titers (FIG. 12E). These results demonstrated that GP nanoparticle-induced immune responses significantly inhibited viral replication in mouse lungs, comparable to H3-CpG mix.

We measured the inflammatory cytokine (TNF-α, IL-12, and IL-6) levels in the BALF samples to evaluate the pulmonary immunopathology. As shown in FIGS. 12F, 12G, and 12H, both naïve and soluble H3-immunized mice produced high levels of TNF-α, IL-12, and IL-6 after virus infection. In comparison, the mice in the GP-H3, GP-H3/CpG, and H3+CpG mix groups displayed significantly reduced TNF-α, IL-12, and IL-6 levels. We also detected substantially higher Aichi virus-specific IgA and IgG antibody levels in BALF samples of infected mice in the GP nanoparticle-immunized groups (FIG. 13B).

These results demonstrated the GP-H3 nanoparticles without an adjuvant provided complete protection in mice against Aichi virus infection. GP nanoparticles showed great promise in boosting the immune responses of influenza HA and providing protection against influenza virus infection, comparable to the model adjuvant CpG. The GP nanoparticles are a potent i.n. vaccine platform to bring recombinant protein vaccines into clinical applications when rare adjuvants are available for this purpose (CpG is still a laboratory adjuvant for vaccination studies at present).

### (6) Protective Efficacy Against Heterologous Influenza Virus Challenge

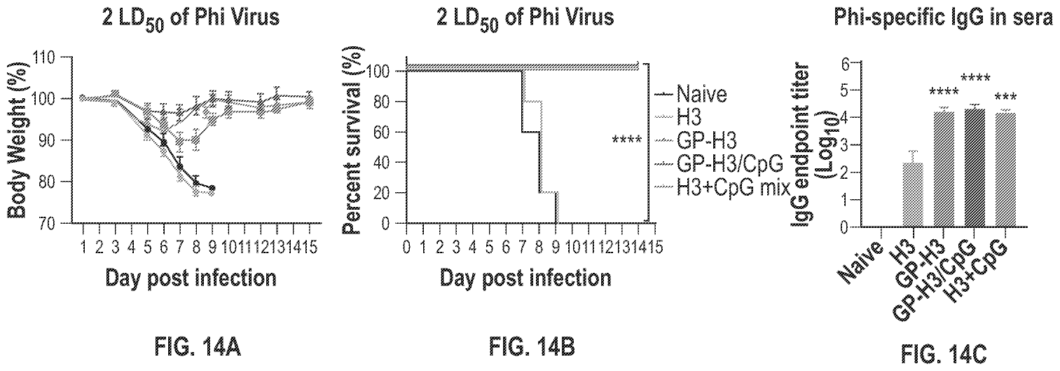

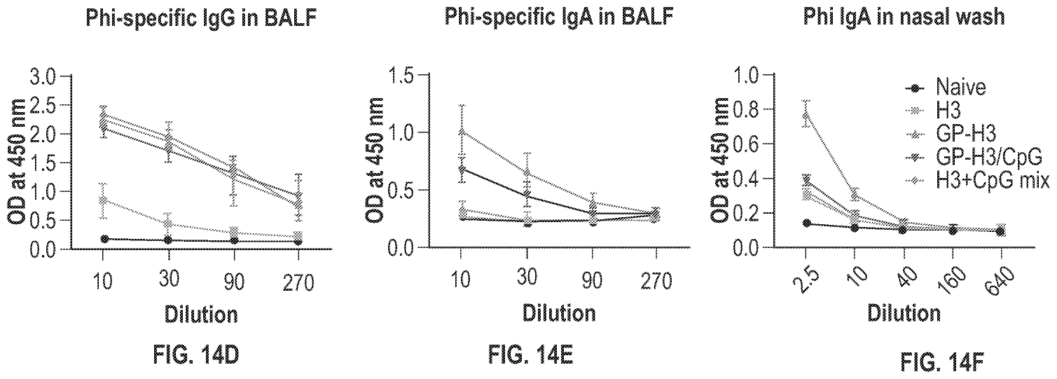

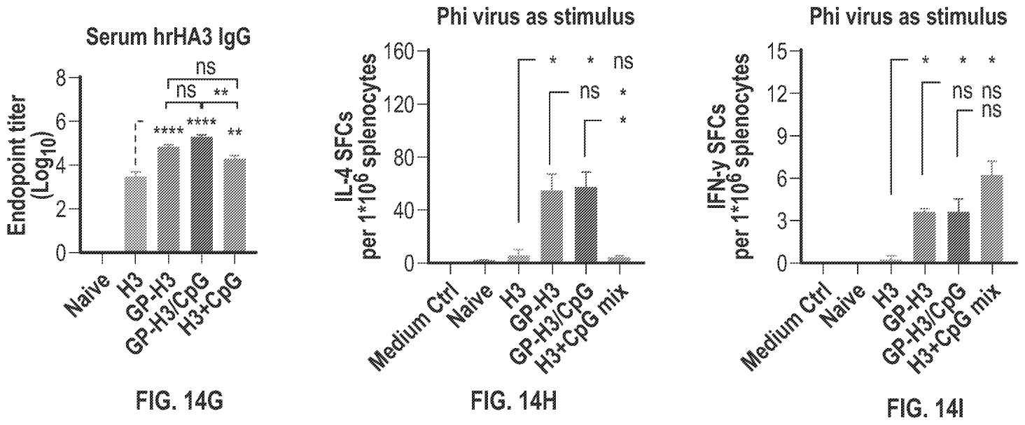

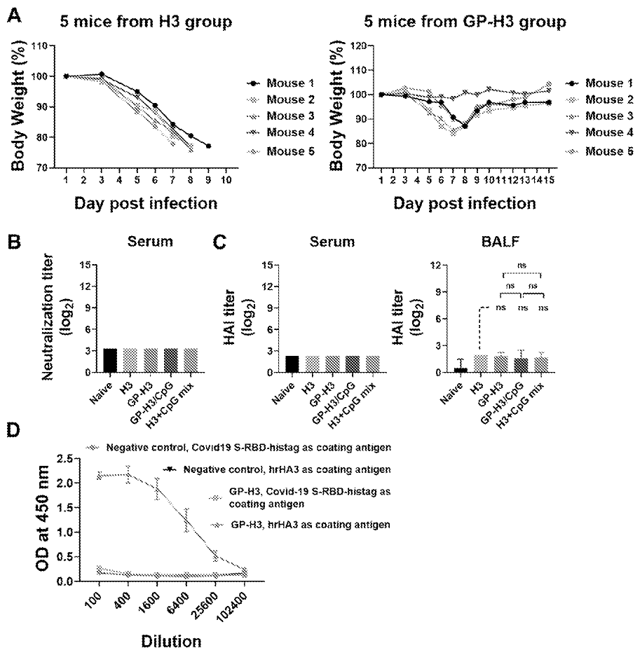

We employed the heterologous A/Philippines/2/1982 (Phi, H3N2) virus to study the cross-protective effect conferred by GP nanoparticle i.n. vaccination. Immunized mice were challenged with 2×LD₅₀ of Phi virus 4 weeks post boosting immunization. As shown in FIG. 14A, 14B, and FIG. 15A, all mice in the soluble H3 group suffered rapid and severe weight loss and died in days 7˜9 post-challenge, the same as the naïve control group. GP-H3, GP-H3/CpG, and H3-CpG mix group showed full protection with slight weight loss.

A comparative analysis of Aic and Phi HA amino acid sequences showed a difference of 8.48%, representing a substantial antigenic drift (Table 3). We investigated the cross-reaction and neutralization activities of mice immune serum and BALF against Phi. As shown in FIG. 14C, GP-H3 and GP-H3/CpG nanoparticles and H3-CpG mix induced significantly higher Phi virus-specific IgG antibody titers than soluble H3. However, no apparent cross-neutralization titers were observed in all groups (FIG. 15B). GP nanoparticles also induced higher Phi virus-specific IgA levels in nasal washes and higher IgG and IgA levels in BALF (FIGS. 14D, 14E, and 14F). Interestingly, the GP-H3 nanoparticle-induced cross-reactive mucosal IgA titers were higher than that in H3+CpG mix group. No noticeable cross-reactive HAI titers against Phi virus were detected in sera or BALF of all groups (FIG. 15C). We measured antibody titers specific to the head-removed Aichi HA stalk protein (hrHA3) and observed significantly higher antibody titers in GP-H3 and GP-H3/CpG nanoparticle groups than the soluble H3 group (FIG. 14G). An Elisa assay using an irrelevant his-tagged SARS-CoV-2 spike protein receptor-binding domain (RBD) as the coating antigen excluded the influence of the his-tag on hrHA3 (FIG. 15D). HA-loaded GP nanoparticles significantly induced HA stalk-specific antibodies, contributing to the heterologous protection against the Phi virus.

TABLE 3
Comparative analysis of the amino acid sequences between influenza A/Aichi/2/1968
HA and A/Philippines/2/1982 HA proteins.

| | |
|---|---|
| (A) | MKTIIALSYIFCLPLGQDLPGNDNSTATLCLGHHAVPNGTLV |
| Amino acid | KTITDDQIEVTNATELVQSSSTGKICNNPHRILDGIDCTLIDA |
| sequence of | LLGDPHCDVFQNETWDLFVERSKAFSNCYPYDVPDYASLR |
| influenza A | SLVASSGTLEFITEGFTWTGVTQNGGSNACKRGPGSGFFSRL |
| virus | NWLTKSGSTYPVLNVTMPNNDNFDKLYIWGIHHPSTNQEQ |
| (A/Aichi/2/1968) | TSLYVQASGRVTVSTRRSQQTIIPNIGSRPWVRGLSSRISIYW |
| hemagglutinin | TIVKPGDVLVINSNGNLIAPRGYFKMRTGKSSIMRSDAPIDT |
| (HA) protein | CISECITPNGSIPNDKPFQNVNKITYGACPKYVKQNTLKLAT |
| | GMRNVPEKQTRGLFGAIAGFIENGWEGMIDGWYGFRHQNS |
| | EGTGQAADLKSTQAAIDQINGKLNRVIEKTNEKFHQIEKEFS |
| | EVEGRIQDLEKYVEDTKIDLWSYNAELLVALENQHTIDLTD |
| | SEMNKLFEKTRRQLRENAEEMGNGCFKIYHKCDNACIESIR |
| | NGTYDHDVYRDEALNNRFQIKGVELKSGYKDWILWISFAIS |
| | CFLLCVVLLGFIMWACQRGNIRCNICI (SEQ ID NO: 1) |
| (B) | MKTIIALSYMFCLVFAQNLPGNDNSTATLCLGHHAVPNGTL |
| Amino acid | VKTITNDQIEVTNATELVQSSSTGRICDSPHRILDGKNCTLID |
| sequence of | ALLGDPHCDGFQNEKWDLFVERSKAFSNCYPYDVPDYASL |
| influenza A | RSLVASSGTLEFINEGFNWTGVTQSGGSYTCKRGSNNSFFSR |
| virus | LNWLYESESKYPVLNVTMPNNGKFDKLYIWGIHHPSTDKE |
| (A/Philippines/2/1982) | QTNLYIRASGRVTVSTKRSQQTVIPNIGSRPWVRGLSSRISIY |
| hemagglutinin | WTIVKPGDILLINSTGNLIAPRGYFKIRTGKSSIMRSDAPIGTC |
| (HA) protein | SSECITPNGSIPNDKPFQNVNKITYGACPRYVKQNTLKLATG |
| | MRNVPEKQTRGIFGAIAGFIENGWEGMVDGWYGFRHQNSE |
| | GTGQAADLKSTQAAIDQINGKLNRVIEKTNEKFHQIEKEFSE |
| | VEGRIQDLEKYVEDTKIDLWSYNAELLVALENQHTIDLTDS |
| | EMNKLFEKTRKQLRENAEDMGNGCFKIYHKCDNACIGSIR |
| | NGTYDHDVYRDEALNNRFQIKGVELKSGYKDWILWISFAIS |
| | CFLLCVVLLGFIMWACQKGNIRCNICI (SEQ ID NO: 2) |
| Difference | 8.48%, as determined by the NCBI protein BLAST tool. |
| between A and | |
| B | |

We evaluated the cytokine-secreting splenocytes. Under the stimulation with inactivated Phi viruses, GP nanoparticle groups showed significantly higher IL-4 and IFN-γ-secreting splenocyte populations (FIGS. 14H and 14I). In contrast, the soluble H3 group demonstrated few such splenocytes.

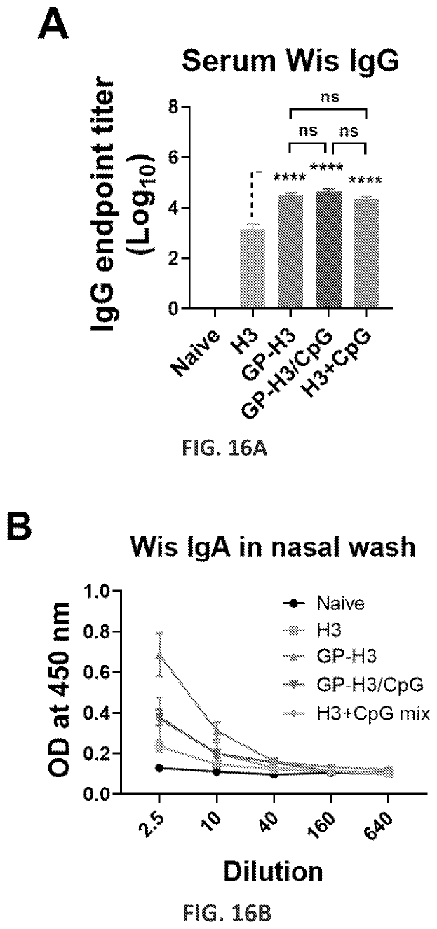

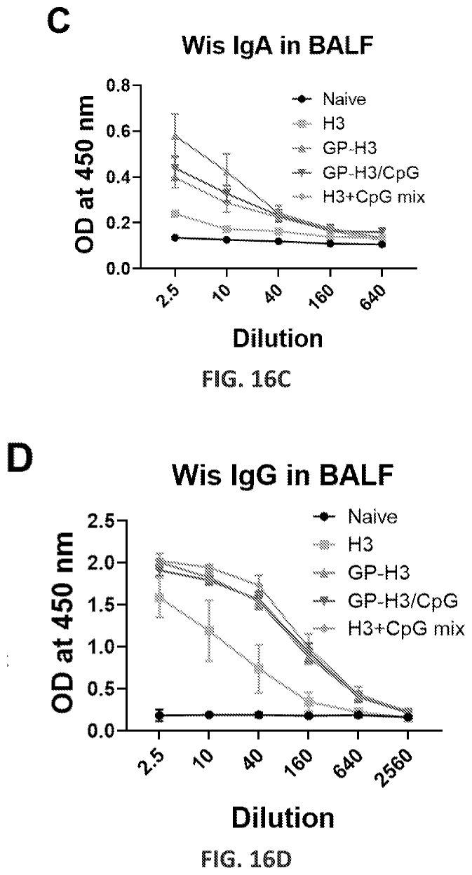

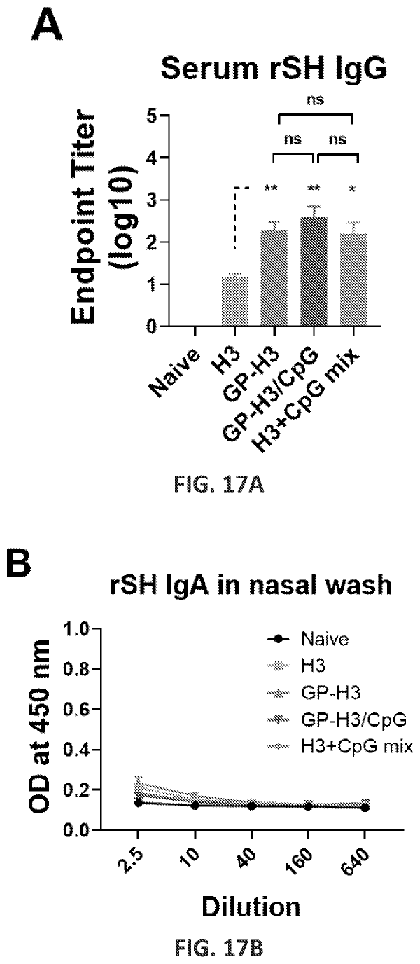

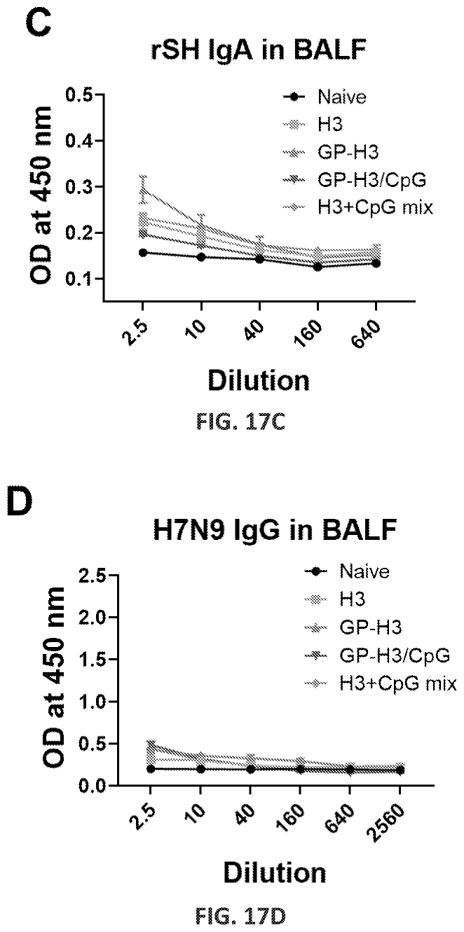

Next, we studied cross-reactive antibody levels in immune sera and mucosal washes against A/Wisconsin/15/2009 (H3N2, Wis) and reassortant A/Shanghai/2/2013 (H7N9, rSH) from HA phylogenic Group 2. GP-H3 and GP-H3/CpG nanoparticles induced significantly higher serum IgG antibodies specific to Wis and rSH than soluble H3, while antibody levels specific to rSH were much lower than that to Wis (FIGS. 16A and 17A). We observed elevated mucosal antibody (IgG and IgA) levels specific to Wis from the GP nanoparticle groups (FIGS. 16B, 16C, and 16D) in contrast to the low or undetectable antibody levels to rSH (FIGS. 17B, 17C, and 17D). The results indicated that GP-H3 nanoparticles can confer better protection against heterologous strains in the same subtype but limited protection against heterosubtypic viruses (such as H7 subtype viruses) even from the same HA group.

### b) Discussion

Broadly protective influenza vaccines are urgently needed due to the continuous antigenic drift and shift of influenza viruses. Influenza mucosal immunity can confer broad cross-protection against heterologous and heterosubtypic viruses. Intranasal vaccination with recombinant HA vaccines is a safe and promising strategy in the generation of mucosal immunity and prevention of influenza virus infection. However, the efficacy of intranasally administrated protein vaccines is challenged by the harsh and tolerogenic nasal epithelium. Nanoparticle-based vaccines can overcome obstacles associated with intranasal vaccine delivery.

Graphene oxide (GO) nanoparticles are a type of two-dimensional sheet-like nanomaterials (i.e., nanosheet) that have demonstrated superior attributes for drug delivery, including ultra-large surface area, easy modification, and excellent physiological biocompatibility. We generated a GO-based influenza vaccine nanoplatform by functionalizing GO with branched polyethylenimine (PEI) for intranasal vaccination. Subsequently, we constructed influenza GO-PEI (GP) nanoparticles in a simple, easily manipulated, and productive mixing/adsorption approach. This vaccine platform possesses multiple features favorable for enhancing intranasal vaccines' immunogenicity, including high antigen loading capacity, mucoadhesive and positive particle surface, and high flexibility for various vaccine components. With immunoenhancing features synergizing in the same particles, GP nanoparticle vaccines facilitate a comprehensive immune response, as seen in the study.

Antibody response to influenza HA is an essential attribute for vaccines designed to prevent influenza virus infection. Enhancing the magnitude and breadth of antibody responses is critical for developing highly efficient and broadly protective influenza vaccines. We found that influenza GP nanoparticles (GP-H3 and GP-H3/CpG) vaccination potently boosted the antibody responses in systemic sites and mucosal surfaces. We observed significantly increased antigen-specific IgG levels, HAI titers, and microneutralization titers in mouse immune sera of influenza GP nanoparticle groups than the soluble H3 group. We also detected substantially enhanced cross-reactive antibody titers against the heterologous Phi and Wis viruses. Moreover, high-level IgG antibodies were induced against the conserved HA stalk antigen, indicating more broad protection. On the other hand, the “antigen reservoir” effect was discovered in GO nanoparticles in previous studies. The cross-reactive IgG antibodies in mouse sera may benefit from the improved antigen sustainability necessary for B cell somatic hypermutation/affinity maturation and class-switching in germinal centers, particularly for weakly immunogenic HA stalk regions. Fc-mediated effector mechanisms, such as antibody-dependent cellular cytotoxicity (ADCC) and antibody-dependent cellular phagocytosis (ADCP), can contribute to cross-protection.

Mucosal immunity driven by IgA is one of the major contributors to influenza virus protection. Secretory IgA (sIgA) is more broadly reactive than IgG. As sIgA is highly effective at preventing influenza infection at the portal of virus entry, the induced sIgA in GP nanoparticle groups is also a critical component of the protective scenario. The sIgA antibodies in the mucosal washes revealed improved antibody binding breadth as well. The cross-reactive IgG antibodies in mouse sera benefit from the improved antigen sustainability necessary for B cell somatic hypermutation/affinity maturation and class-switching in germinal centers. As an outcome, the improved antibody breadth conferred the increased cross-protection efficacy in GP nanoparticle-immunized mice.

Cellular responses play crucial roles in the fight against influenza virus infections. T helper cells are the central players in organizing an effective immune response. The differentiation of naive helper T cells into subtypes is programmed by a specific cytokine niche. TNF-α, as one of the most critical proinflammatory cytokines in cellular immunity, attracts the migration of immune cells, enhancing immune responses. IL-6 can promote the production of IL-4, a potent cytokine that directs Th2 differentiation. We found that influenza GP nanoparticles boosted the output of TNF-α and IL-6 in JAWS II dendritic cell cultures. Moreover, the nanoparticle vaccination dramatically increased the generation of IL-4-secreting cells in mouse spleens and cervical lymph nodes. IL-4 can facilitate the proliferation and differentiation of B cells into ASCs. These results were in agreement with the elevated antigen-specific IgG and IgA ASC populations observed. IFN-γ is critical in modulating cellular immunity and coordinating numerous protective functions in virus infection. We also observed significantly more IFN-γ-producing lymphocytes, which contributed to both homologous and heterologous protection in GP nanoparticle vaccination.

We observed strong cellular responses by GP nanoparticle immunization, including CD8⁺ T cell responses. Intracellular cytoplasmatic antigen localization is a prerequisite for the cross-presentation of extracellular antigens to CD8⁺ T cells by MHC I molecules. GO-based nanoparticles can specifically traffic through an intracellular cytosolic pathway because of the capability to destabilize intracellular vesicle lipid membranes or via GO-triggered autophagy. Moreover, it is well-documented that PEI could induce endosomal escape because of the “proton sponge effect”. These intriguing physicochemical characteristics of GP nanoparticles contribute to the CD8 T cell responses and the heterologous protection observed.

As vaccine delivery carriers, GP nanoparticles can simultaneously deliver antigens and adjuvants. The large surface area and high loading capability of GP nanoparticles facilitated multiple antigens displayed on the surface, resulting in strong interaction with immune cells through multivalent recognitions. In addition to the role as a delivery carrier, GP nanoparticles exhibited immunostimulating effects. We demonstrated that GP nanoparticles significantly promoted antigen internalization in dendritic cells, boosted the proinflammatory cytokine production, and stimulated DC maturation. We observed that antigen-free GP nanoparticles enhanced DCs generation of IL-6 and expression of CD86, indicating an inherent immunostimulating effect of the particles themselves. Because the GO can destabilize intracellular vesicle lipid membranes (such as endolysosome) and initiate damage-associated molecular pattern (DAMP) innate signaling cascades, the signaling pathway activation partially contribute to the observed immunostimulating effects of GP nanoparticles. Otherwise, the GP nanoparticle adjuvant effect deserved to be studied in the future.

CpG ODN is an acknowledged potent mucosal immunomodulator. Despite the excellent adjuvanticity, CpG has not been approved for human use due to the safety concerns, such as activating autoreactive B cells and increasing the risk of autoimmune disease and the variable magnitude of immune effects in clinical trials. In the absence of any additional adjuvant, the GP nanoparticle significantly boosted antigen-specific immune responses, conferring complete protection in mice against influenza infection, comparable to the role of CpG in the H3+CpG mix. The GP nanoparticles can function as a self-adjuvanted vaccine platform. The strong self-adjuvant effect of GP nanoparticles masked the role of CpG co-incorporated (GP-H3 vs. GP-H3/CpG).

In summary, we generated novel influenza HA-GO nanoparticles for high-performance intranasal influenza vaccination. The functionalized GP nanoparticle served as a robust delivery/adjuvant system, significantly enhancing the recombinant HA immunogenicity. Immunization with influenza GP nanoparticles significantly boosted the magnitude and breadth of immune responses, conferring both homo- and heterologous protection. The GP nanoparticle are a strong candidate for developing intranasal vaccines. With high versatility and flexibility, the GP nanoparticles can be easily adapted for constructing mucosal vaccines for different respiratory infectious pathogens.

### c) Experimental Section

### (1) Experimental Design

The objective of this study was to develop a new generation of safe, efficient, and virus-independent influenza intranasal vaccines that induce cross-protection. To this end, we developed graphene oxide-based recombinant influenza HA nanoparticle vaccines. To validate the immunoenhancing effects of these vaccines, we studied their interaction with JAWS II dendric cells. We further evaluated the immune responses and prophylaxis protection against both homologous and heterologous influenza strains in mice after intranasal immunization with these nanoparticles. The number of mice per experimental group is indicated in the method. Antibody analysis and challenge experiments were repeated twice. Statistical analyses were conducted when applicable and were included in the figure legends. All animal experiments were performed in compliance with the Institutional Animal Care and Use Committee (IACUC) guidelines of Georgia State University.

### (2) Materials

GO powder, branched PEI with an average molecular weight of 60 kDa, and N-(3-dimethyl aminopropyl-N-ethylcarbodiimide) hydrochloride (EDC) were purchased from Sigma-Aldrich. CpG ODN1826 was bought from InvivoGen. 5(6)-Carboxyfluorescein diacetate N-succinimidyl ester (CFSE) and bis[sulfosuccinimidyl] suberate (BS3) were bought from Invitrogen, USA.

### (3) Cell Lines and Viruses

Spodoptera frugiperda (Sf9, ATCC, CRL-1711) insect cells, JAWS II murine dendritic cells (ATCC® CRL-11904™), and Madin-Darby Canine Kidney cells (MDCK (NBL-2), ATCC® CCL-34™) were grown in conditions recommended by the vendor.

A/Aichi/2/1968 (Aic, H3N2) and A/Philippines/2/1982 (Phi, H3N2) A/Wisconsin/15/2009 (Wis, H3N2), and reassortant A/Shanghai/2/2013 (rSH, H7N9) influenza viruses were expanded in embryonated chicken eggs. Mouse-adapted Aic and Phi were prepared as lung homogenates from intranasally (i.n.) infected mice. The median lethal dose (LD₅₀) was determined by the method of Reed and Muench.

### (4) Expression, Purification, and Characterization of Recombinant Trimeric HA

GCN4-stabilized Aichi HA (H3) was expressed in Sf9 cells by a Bac-to-Bac baculovirus expression system (Invitrogen, USA) and purified by a His-tag affinity method using Ni-NTA resins. SDS-PAGE followed with Coomassie Blue (Bio-Rad, USA) staining and Western blots using anti-Aichi HA antibodies was performed to verify the purified H3. The H3 concentration was determined by a BCA assay kit (Thermo Fisher Scientific, USA). HA trimerization was determined by BS3 crosslinking at different concentrations (0, 0.5, 5, and 10 mM) followed by Western blots.

### (5) Preparation of Graphene Oxide (GO) and GO-PEI (GP) Nanoparticles

GO nanoparticles were prepared from GO powder by tip ultrasonication. Briefly, GO powder was resuspended in Milli-Q water and then sonicated at 100 W in an ice-bath for 2 h. GO particle size was measured at different sonication time intervals by dynamic light scattering (DLS) with a Malvern Zetasizer Nano ZS (Malvern Instruments, USA). The final GO solution was centrifuged at 6000 rpm for 5 min to remove larger particles.

We prepared GO-PEI nanoparticles by using Carbodiimide coupling method. 10 mL of GO solution (0.5 mg/mL) was sonicated for 30 min, and then 0.6 mL of PEI solution (50 mg/mL) was added and sonicated for another 30 min. The mixture was activated by adding two batches of EDC (50 mg) at an interval of 30 min followed by stirring at room temperature overnight. GO-PEI nanoparticles were collected by centrifugation at 15,000 rpm at 4° C. for 2 h and then repeatedly washed with Milli-Q water to remove the free PEI. GO-PEI nanoparticle pellets were resuspended in pure water and stored at 4° C. in dark for later use. The concentrations of GO and GO-PEI nanoparticles were determined by their absorbances at 230 nm with a Nanodrop spectrometer (Thermo Fisher Scientific, USA), according to the Beer-Lambert law (A=εlc). Herein, the mass extinction coefficient (ε) of GO was set as 65 mL mg⁻¹ cm⁻¹.

### (6) Fabrication and Optimization of GP-H3 and GP-H3/CpG Nanoparticles

GP-based vaccine formulations were prepared by a simple mixing/adsorption approach. The loading capability of H3 on the GP nanoparticles was evaluated via reducing SDS-PAGE. We prepared a series of GP-H3 nanoparticles at six different weight ratios (2:1, 1:1, 1:2, 1:4, 1:6, 1:8) under stirring for 20 min at room temperature. The GP-H3 nanoparticles were collected by centrifugation at 15,000 rpm for 20 min and then dispersed in an equal amount of PBS (nanoparticles). The supernatants were kept separately (supernatants). Both the nanoparticles and supernatants for all the formulations were analyzed by 10% SDS-PAGE. The gel was stained with Coomassie blue and imaged with the ChemiDoc Touch imaging system (Bio-Rad).

In some formulations, CpG was co-loaded onto GP nanoparticles with antigens. We employed agarose gel electrophoresis to investigate whether all the feeding CpG were complexed and loaded onto the GP nanoparticles. Soluble CpG, GP-H3/CpG (10:5:1), GP-H3/CpG (10:5:2.5), and H3+CpG mix, were centrifuged at 15000 rpm for 20 min, and the supernatants were analyzed by 1% agarose gel electrophoresis. The gel was visualized with ChemiDoc Touch imaging system (Bio-Rad) to determine the existence of CpG in the supernatants.

### (7) Nanoparticle Characterization

Particle sizes and Zeta potentials of the resulting nanoparticles were measured by DLS. The GO nanoparticle morphology was characterized by atomic force microscopy (AFM) with a Bruker Icon AFM and transmission electron microscopy (TEM) with a JEOL 100 CX-II. UV-Vis absorption spectra of the samples were recorded by a Nanodrop spectrometer (Thermo Fisher Scientific, USA). The thermogravimetric analysis (TGA) of the GO and GP nanoparticles was performed using a TA Q500 instrument under an inert nitrogen atmosphere. The heating rate and nitrogen flow rate were 10° C./min and 50 mL/min, respectively. The maximum temperature was 600° C.

### (8) Cellular Uptake in JAWS II Cells by Immunofluorescence Imaging

The internalization profiles of soluble H3 and GP-H3 nanoparticles in JAWS II cells were studied by immunofluorescence (IF) imaging. JAWS II cells were seeded at 2×10⁵ cells/well (2×10⁵ cells/mL, 1 mL) in a 24-well cell culture plate and treated with soluble H3 or GP-H3 nanoparticles at an H3 concentration of 10 μg/mL. Untreated cells were used as negative controls. After 16-h incubation, the cells were washed twice with DPBS, and then fixed and permeabilized with BD fixation/permeabilization buffer at 4° C. for 20 min, following by blocking with 5% BSA for 1 h at room temperature. After washing twice more, the cells were stained successively with antigen-specific Abs for 1 h, and fluorescent DyLight™ 649-conjugated goat anti-mouse IgG antibodies (Biolegend, USA) for 30 min. The cells were recorded by using a Keyence BZ-X710 fluorescence microscope.

### (9) Proinflammatory Cytokine Profiles and Maturation of Stimulated JAWS II Cells

JAWS II cells were seeded at 4×10⁴ cells/well (4×10⁵ cells/mL, 100 μL/well) in a 96-well cell culture plate, followed by treatment with soluble H3, GP, GP-H3, GP-H3/CpG, or H3+CpG mix formulations for 16 h. The final concentration of H3 was 5 or 10 μg/mL. The ratio of GP:H3:CpG was 10:5:1 (w:w:w). Untreated cells were used as negative controls. Supernatants were collected for determining IL-6 and TNF-α levels by using cytokine enzyme-linked immunosorbent assay (ELISA) kits (Thermo Scientific). Cells were collected for evaluating the maturation of stimulated JAWS II cells. The cell surface marker CD86 was determined by flow cytometry. Data were analyzed with the FlowJo software.

### (10) Immunization, Sample Collection, and Challenge

Female BALB/c mice (6-8 weeks old, n=20) were intranasally (i.n.) immunized with 30 μL of soluble H3, GP-H3, GP-H3-CpG or H3+CpG in saline (5 μg of H3 protein per mouse). Mice were vaccinated twice at an interval of 4 weeks.

Prime sera were collected 3 wks post priming immunization, and boost sera were collected 3 wks post-boosting immunization (n=7). Nasal washes and bronchoalveolar lavage fluid (BALF) (n=4) were collected three weeks after boosting immunization by flushing the respective cavities with 1 mL of cold sterile PBS, and then centrifuged at 10,000 rpm for 5 min. Spleens, cervical lymph nodes (CLNs), and nasal-associated lymphoid tissues (NALTs) of the immunized mice (n=4) were isolated three weeks after boosting immunization. Single-cell suspensions were obtained by gently grinding tissues with frosted microscope slides. Splenocytes were harvested after treating with RBC lysis buffer.

Four weeks after the boosting immunization, mice (n=5) were intranasally challenged with 15×LD₅₀ of mouse-adapted Aichi or 2×LD₅₀ of Philippines virus in 30 μL saline. Mouse body weight loss and survival rates were recorded daily for 2 weeks post-infection. A weight loss exceeding 20% was used as a humane endpoint. Five days post-infection with Aichi virus, mice were sacrificed, and lung tissues were isolated for determining lung viral titers and histological analysis.

### (11) Safety Evaluation of GP-H3 Nanoparticles

Mouse body weight was monitored for 7 days post-vaccination. Histological examination of mouse nasal mucosa and lung tissues was performed with H&E staining 24 hours and 7 days post-vaccination, respectively. The nasal cavities were decalcified with ethylene diamine tetraacetic acid (EDTA) disodium salt solution, followed by cryosectioning and Haemotoxylin and Eosin (H&E) staining. Lung tissues were paraffin-embedded, followed by sectioning and H&E staining.

### (12) Antibody ELISA, Hemagglutination-Inhibition (HAI) Assay and Viral Neutralization Assay

Antibody ELISA was performed. The virus (Aic, Phi, Wis, rSH) or H3-specific IgG, IgA, or IgE antibody endpoint titers in sera, nasal washes, or BALF samples post-immunization were tested. The highest dilution with an OD₄₅₀ twice that of the naive group was used as the endpoint titer. HAI titers of mouse boost immune sera were determined. Sera samples were pre-treated with receptor destroying enzyme (RDE II, Denka Seiken Co., Ltd) overnight at 37° C. and then heat-inactivated at 56° C. for 30 min before the test. Turkey red blood cells (0.5%) were used for this assay, and the highest dilution able to inhibit virus hemagglutination was used as the HAI titer.

The median tissue culture infective doses (TCID₅₀) of Aichi and Philippines viruses were determined according to Reed and Muench method. Two-fold serial dilutions of heat-inactivated (56° C. for 30 min) mouse boost immune sera in EMEM (50 μL) were mixed with 100-fold TCID₅₀ of Aichi or Philippines virus in EMEM (50 μL) for 2 h at 37° C. After incubation, the mixture was added to MDCK cell monolayers (100 μL/well, 1.5×10⁵ cells/mL, with 2 μg/mL of TBCK-trypsin), and incubated for 3 days at 37° C. A standard hemagglutination assay was used to determine virus inhibition.

### (13) Enzyme-Linked Immunospot (ELISpot) Assay

Cytokine ELISpot assay (BioLegend, USA) was performed to analyze IL-4 or IFN-γ secreting cells. Briefly, splenocytes or CLN cells (3×10⁶ cells/mL, 100 μL/well) were seeded into 96-well filtration plates (MultiScreen™-HA, Millipore) that were pretreated with anti-mouse IFN-γ or IL-4 antibodies, and then stimulated with H3 (4 μg/mL) for 24 h at 37° C. After removing cells, plates were incubated with biotin-conjugated IFN-γ or IL-4 detection antibody at 37° C. for 1 h, followed by the addition of HRP-streptavidin for another 1 h. True Blue Peroxidase substrate (KPL) was used to develop spots, and spots were recorded with Bioreader-6000-E (BIOSYS).

B-cell ELISpot assay was used to evaluate the antigen-specific antibody-secreting cells (ASCs). Briefly, 96-well filtration plates were precoated with H3 proteins (50 μL/well, 4 μg/mL) overnight at 4° C., washed, blocked, and then splenocytes or NALT cells (3×10⁶ cells/mL, 100 μL/well) were seeded and incubated overnight at 37° C. After removing cells, HRP-conjugated anti-mouse IgG or IgA antibodies were added for 1 h at room temperature. True Blue Peroxidase substrate was used to develop spots. Results were recorded with Bioreader-6000-E.

### (14) Proliferation Assay by CFSE Staining

The proliferation ability of splenocytes was evaluated by using the CFSE Cell Proliferation Assay Kit (Invitrogen, USA). Splenocytes were stained with CFSE at 37° C. for 10 min, washed with complete RPMI 1640 medium thoroughly, and then seeded into 24-well plates (1×10⁶ cells/well) and incubated with H3 (5 μg/mL) for 60 h. The cells were then harvested and stained for 30 min at 4° C. with anti-mouse PE-Cy7-anti-CD4 and PE-Cy5-anti-CD8α antibodies (Biolegend, USA) in the presence of Fc blocker. After washing and resuspending in FACS buffer (PBS+5% FCS), the cells were measured with a BD LSRFortessa flow cytometer (BD biosciences). Data were analyzed using the FlowJo software (FlowJo LLC). The splenocytes from naïve mice were used as control.

### (15) Analysis of T Cell Subpopulations

The CD3⁺CD4⁺ and CD3⁺CD8⁺ T cell subpopulations in spleens of immunized mice were analyzed by FACS. Briefly, splenocyte suspensions (1×10⁶ cells/mL, 1 mL/well) were seeded into 24-well plates and re-stimulated with H3 (5 μg/mL) for 36 h at 37° C. The cells were harvested and stained for 30 min at 4° C. with PE-anti-CD3E, PE-Cy7-anti-CD4, and PE-Cy5-anti-CD8α antibodies. After washing and resuspending in FACS buffer, the cells were detected with a BD LSRFortessa flow cytometer and analyzed with the FlowJo software.

(16) Histological Analysis after Virus Infection

On day 5 post-infection with Aichi virus, mice were euthanized. Lung tissues were isolated, fixed with 10% neutral buffered formalin overnight at 4° C., dehydrated, and then embedded in paraffin, followed by sectioning and Haemotoxylin and Eosin (H&E) staining. The tissue sections were recorded by a Keyence BZ-X710 microscope and examined by five unbiased pathologists. The degree of leukocyte infiltration was scored on a scale of 0 to 5. Scores were given as absent (0), subtle (1), mild (2), moderate (3), severe (4), and massive (5).

### (17) Lung Viral Titration

Five days post-infection with Aichi virus, lung tissues were homogenized and supernatants were cleared by centrifugation at 1000 rpm for 10 min at 4° C. Ten-fold serial dilutions of lung supernatants (100 μL) were added to the prepared 96-well plates containing MDCK cells (1.5×10⁵ cells/mL, 100 μL), and co-cultured for 5 days. A standard hemagglutination assay was carried out to determine viral titers in the supernatants by the Reed-Munchen method.

### (18) Evaluation of Inflammatory Cytokine Levels in BALF

To evaluate the pulmonary immunopathology caused by virus infection, inflammatory cytokine (TNF-α, IL-12, and IL-6) levels in the BALF of infected mice were measured according to the cytokine ELISA kits instructions (Thermo Scientific).

### (19) Statistical Analysis

All data were represented as means with the standard error of the mean (SEM). Statistical significance was analyzed by the one-way analysis of variance (ANOVA). A probability value (p) of less than 0.05 is considered to be statistically significant. P<0.05 (*), p<0.01 (**), p<0.001 (***), p<0.0001 (****), p>0.05 (ns). Survival rate statistical analysis was performed with the Log-rank (Mantel-Cox) test. The analysis was performed with GraphPad Prism 8 program (GraphPad software).

### F. References

Asahi-Ozaki, Y. et al. Secretory IgA antibodies provide cross-protection against infection with different strains of influenza B virus. J. Med. Virol. 74, 328-335 (2004).
Burgdorf, S., Scholz, C., Kautz, A., Tampe, R. & Kurts, C. Spatial and mechanistic separation of cross-presentation and endogenous antigen presentation. Nat. Immunol. 9, 558-566 (2008).
Cao, Y. et al. Ultrasmall graphene oxide supported gold nanoparticles as adjuvants improve humoral and cellular immunity in mice. Adv. Funct. Mater. 24, 6963-6971 (2014).
Chevalier, C. & Calzas, C. Innovative mucosal vaccine formulations against influenza A virus infections. Front. Immunol. 10, 1605 (2019).
Demirel, M. C., Vural, M. & Terrones, M. Composites of proteins and 2D nanomaterials. Adv. Funct. Mater. 28, 1704990 (2018).
Deng, L. et al. Double-layered protein nanoparticles induce broad protection against divergent influenza A viruses. Nat. Commun. 9, 1-12 (2018).
Dykman, L. A. Gold nanoparticles for preparation of antibodies and vaccines against infectious diseases. Expert Rev. Vaccines, 1-13 (2020).
Feng, L., Zhang, S. & Liu, Z. Graphene based gene transfection. Nanoscale 3, 1252-1257 (2011).
Fromen, C. A. et al. Controlled analysis of nanoparticle charge on mucosal and systemic antibody responses following pulmonary immunization. Proc. Natl. Acad. Sci. U.S.A. 112, 488-493 (2015).
Gause, K. T. et al. Immunological principles guiding the rational design of particles for vaccine delivery. ACS nano 11, 54-68 (2017).
Georgakilas, V. et al. Noncovalent functionalization of graphene and graphene oxide for energy materials, biosensing, catalytic, and biomedical applications. Chem. Rev. 116, 5464-5519 (2016).
Hopkins, R. J. et al. Randomized, double-blind, placebo-controlled, safety and immunogenicity study of 4 formulations of Anthrax Vaccine Adsorbed plus CPG 7909 (AV7909) in healthy adult volunteers. Vaccine 31, 3051-3058 (2013).
Impagliazzo, A. et al. A stable trimeric influenza hemagglutinin stem as a broadly protective immunogen. Science 349, 1301-1306 (2015).
Kanekiyo, M. et al. Self-assembling influenza nanoparticle vaccines elicit broadly neutralizing HIN1 antibodies. Nature 499, 102-106 (2013).
Krammer, F. The human antibody response to influenza A virus infection and vaccination. Nat. Rev. Immunol. 19, 383-397 (2019).
Krieg, A. M. & Vollmer, J. Toll-like receptors 7, 8, and 9: linking innate immunity to autoimmunity. Immunol. Rev. 220, 251-269 (2007).
Kurapati, R. et al. Dispersibility-dependent biodegradation of graphene oxide by myeloperoxidase. Small 11, 3985-3994 (2015).
Lavelle, E. Generation of improved mucosal vaccines by induction of innate immunity. Cell. Mol. Life Sci. 62, 2750-2770 (2005).
Li, H. et al. Spontaneous protein adsorption on graphene oxide nanosheets allowing efficient intracellular vaccine protein delivery. ACS Appl. Mater. Interfaces 8, 1147-1155 (2016).
Li, S. et al. Polyethylenimine-modified fluorescent carbon dots as vaccine delivery system for intranasal immunization. ACS Biomater. Sci. Eng. 4, 142-150 (2018).
Lin, Y.-L. et al. A CpG-adjuvanted intranasal enterovirus 71 vaccine elicits mucosal and systemic immune responses and protects human SCARB2-transgenic mice against lethal challenge. Sci. Rep. 8, 1-11 (2018).
Meng, C. et al. Graphene oxides decorated with camosine as an adjuvant to modulate innate immune and improve adaptive immunity in vivo. ACS nano 10, 2203-2213 (2016).
Nochi, T. et al. Nanogel antigenic protein-delivery system for adjuvant-free intranasal vaccines. Nat. Mater. 9, 572-578 (2010).
Ohmit, S. E., Petrie, J. G., Cross, R. T., Johnson, E. & Monto, A. S. Influenza hemagglutination-inhibition antibody titer as a correlate of vaccine-induced protection. The J. Infect. Dis. 204, 1879-1885 (2011).
Padilla-Quirarte, H. O., Guerrero, D. V. L., Gutierrez-Xicotencatl, M. L. & Esquivel-Guadarrama, F. R. Protective antibodies against influenza proteins. Front. Immunol. 10, 1677 (2019).
Rajao, D. S. & Perez, D. R. Universal vaccines and vaccine platforms to protect against influenza viruses in humans and agriculture. Front. Microbiol. 9, 123 (2018).
Rohiwal, S. et al. Polyethylenimine based magnetic nanoparticles mediated non-viral CRISPR/Cas9 system for genome editing. Sci. Rep. 10, 1-12 (2020).
Rose, M. A., Zielen, S. & Baumann, U. Mucosal immunity and nasal influenza vaccination. Expert Rev. Vaccines 11, 595-607 (2012).
Rynkiewicz, D. et al. Marked enhancement of the immune response to BioThrax® (Anthrax Vaccine Adsorbed) by the TLR9 agonist CPG 7909 in healthy volunteers. Vaccine 29, 6313-6320 (2011).
Saito, T. et al. Effective collaboration between IL-4 and IL-21 on B cell activation. Immunobiology 213, 545-555 (2008).
Scheiermann, J. & Klinman, D. M. Clinical evaluation of CpG oligonucleotides as adjuvants for vaccines targeting infectious diseases and cancer. Vaccine 32, 6377-6389 (2014).
Sia, Z. R., Miller, M. S. & Lovell, J. F. Engineered Nanoparticle Applications for Recombinant Influenza Vaccines. Mol. Pharm. (2020).
Sinha, A. et al. Carbohydrate-functionalized rGO as an effective cancer vaccine for stimulating antigen-specific cytotoxic T cells and inhibiting tumor growth. Chem. Mater. 29, 6883-6892 (2017).
Smith, W., Andrewes, C. H. & Laidlaw, P. P. A virus obtained from influenza patients. Lancet, 66-68 (1933).
Suzuki, T. et al. Relationship of the quaternary structure of human secretory IgA to neutralization of influenza virus. Proc. Natl. Acad. Sci. U.S.A 112, 7809-7814 (2015).
Tam, H. H. et al. Sustained antigen availability during germinal center initiation enhances antibody responses to vaccination. Proc. Natl. Acad. Sci. U.S.A. 113, E6639-E6648 (2016).
Tu, Y. et al. Destructive extraction of phospholipids from Escherichia coli membranes by graphene nanosheets. Nat. Nanotechnol. 8, 594 (2013).
Vyas, J. M., Van der Veen, A. G. & Ploegh, H. L. The known unknowns of antigen processing and presentation. Nat. Rev. Immunol. 8, 607-618 (2008).
Wang, C., Zhu, W., Luo, Y. & Wang, B.-Z. Gold nanoparticles conjugating recombinant influenza hemagglutinin trimers and flagellin enhanced mucosal cellular immunity. Nanomedicine 14, 1349-1360 (2018).
Wang, J. et al. Pulmonary surfactant-biomimetic nanoparticles potentiate heterosubtypic influenza immunity. Science 367 (2020).
Wang, L. et al. Coated protein nanoclusters from influenza H7N9 HA are highly immunogenic and induce robust protective immunity. Nanomedicine 13, 253-262 (2017).
Wang, L. et al. Nanoclusters self-assembled from conformation-stabilized influenza M2e as broadly cross-protective influenza vaccines. Nanomedicine 10, 473-482 (2014).
Wegmann, F. et al. Polyethyleneimine is a potent mucosal adjuvant for viral glycoprotein antigens. Nat. Biotechnol. 30, 883-888 (2012).
Weldon, W. C. et al. Enhanced immunogenicity of stabilized trimeric soluble influenza hemagglutinin. PLoS One 5, e12466 (2010).
Woodrow, K. A., Bennett, K. M. & Lo, D. D. Mucosal vaccine design and delivery. Annu. Rev. Biomed. Eng. 14, 17-46 (2012).
Xu, L. et al. Functionalized graphene oxide serves as a novel vaccine nano-adjuvant for robust stimulation of cellular immunity. Nanoscale 8, 3785-3795 (2016).
Yang, K. et al. In vivo biodistribution and toxicology of functionalized nano-graphene oxide in mice after oral and intraperitoneal administration. Biomaterials 34, 2787-2795 (2013).
Yue, H. et al. Exploration of graphene oxide as an intelligent platform for cancer vaccines. Nanoscale 7, 19949-19957 (2015).

### G. Sequences

Amino acid sequence for A/Aichi/2/1968 HA protein
SEQ ID NO: 1
MKTIIALSYIFCLPLGQDLPGNDNSTATLCLGHHAVPNGTLVKTITDDQIEVTNATELVQ
SSSTGKICNNPHRILDGIDCTLIDALLGDPHCDVFQNETWDLFVERSKAFSNCYPYDVPD
YASLRSLVASSGTLEFITEGFTWTGVTQNGGSNACKRGPGSGFFSRLNWLTKSGSTYPV
LNVTMPNNDNFDKLYIWGIHHPSTNQEQTSLYVQASGRVTVSTRRSQQTIIPNIGSRPWV
RGLSSRISIYWTIVKPGDVLVINSNGNLIAPRGYFKMRTGKSSIMRSDAPIDTCISECITPN
GSIPNDKPFQNVNKITYGACPKYVKQNTLKLATGMRNVPEKQTRGLFGAIAGFIENGWE
GMIDGWYGFRHQNSEGTGQAADLKSTQAAIDQINGKLNRVIEKTNEKFHQIEKEFSEVE
GRIQDLEKYVEDTKIDLWSYNAELLVALENQHTIDLTDSEMNKLFEKTRRQLRENAEE
MGNGCFKIYHKCDNACIESIRNGTYDHDVYRDEALNNRFQIKGVELKSGYKDWILWISF
AISCFLLCVVLLGFIMWACQRGNIRCNICI
Amino acid sequence for A/Philippines/2/1982 HA protein
SEQ ID NO: 2
MKTIIALSYMFCLVFAQNLPGNDNSTATLCLGHHAVPNGTLVKTITNDQIEVTNATELV
QSSSTGRICDSPHRILDGKNCTLIDALLGDPHCDGFQNEKWDLFVERSKAFSNCYPYDVP
DYASLRSLVASSGTLEFINEGFNWTGVTQSGGSYTCKRGSNNSFFSRLNWLYESESKYP
VLNVTMPNNGKFDKLYIWGIHHPSTDKEQTNLYIRASGRVTVSTKRSQQTVIPNIGSRP
WVRGLSSRISIYWTIVKPGDILLINSTGNLIAPRGYFKIRTGKSSIMRSDAPIGTCSSECITP
NGSIPNDKPFQNVNKITYGACPRYVKQNTLKLATGMRNVPEKQTRGIFGAIAGFIENGW
EGMVDGWYGFRHQNSEGTGQAADLKSTQAAIDQINGKLNRVIEKTNEKFHQIEKEFSE
VEGRIQDLEKYVEDTKIDLWSYNAELLVALENQHTIDLTDSEMNKLFEKTRKQLRENAE
DMGNGCFKIYHKCDNACIGSIRNGTYDHDVYRDEALNNRFQIKGVELKSGYKDWILWI
SFAISCFLLCVVLLGFIMWACQKGNIRCNICI

1. A composition comprising i) a functionalized graphene oxide (GO) nanoparticle comprising polyethyleneimine (PEI) (GO-PEI, GP) and ii) a microbial antigen, vaccine, and/or pharmaceutical agent.

2. The composition of claim 1, wherein the functionalized graphene oxide (GO) nanoparticle comprising polyethyleneimine (PEI) (GO-PEI, GP) is PEGylated.

3. The composition of claim 1, wherein the functionalized graphene oxide (GO) nanoparticle forms a nanosheet.

4. The composition of claim 1, wherein the microbial antigen comprises a peptide, polypeptide, protein, inactivated virus, or heat killed virus.

5. The composition of claim 1, wherein the microbial antigen comprises a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

6. The composition of claim 5, wherein the viral antigen comprises a peptide, polypeptide, or protein of an influenza virus hemagglutinin (HA) protein.

7. (canceled)

8. (canceled)

9. The composition of claim 6, wherein the HA protein comprises a trimeric HA.

10. The composition of claim 5, wherein the viral antigen comprises a peptide, polypeptide, or protein of a coronavirus spike protein or envelope protein; a peptide, polypeptide, or protein of a human immunodeficiency virus (HIV) glycoprotein (gp), envelope protein (Env), or gag protein; a peptide, polypeptide, or protein of a zika virus capsid protein, envelope protein, or premembrane/membrane protein; a peptide, polypeptide, or protein of a dengue virus capsid protein, envelope protein, or premembrane/membrane protein; a peptide, polypeptide, or protein of a Herpes Simplex (HSV)-1 or HSV-2 glycoprotein D or glycoprotein G: a peptide, polypeptide, or protein of a varicella zoster virus (VZV) glycoprotein E; a peptide, polypeptide, or protein of an Epstein-Barr virus (EBV) or Cytomegalovirus (CMV) glycoprotein; a peptide, polypeptide, or protein of a Respiratory synsyctial virus (RSV) G protein; a peptide, polypeptide, or protein of a measles virus hemagglutinin protein or fusion protein; a peptide, polypeptide, or protein of a rabies virus glycoprotein; a peptide, polypeptide, or protein of a rotavirus viral protein 4 or viral protein 7; a peptide, polypeptide, or protein of a hepatitis B virus surface antigen; or a peptide, polypeptide, or protein of a hepatitis C virus E1, E2, or E3 protein.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The composition of claim 1, wherein the microbial antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

24. The composition of claim 1, wherein the pharmaceutical agent comprises zanamivir, oseltamivir, peramivir, baloxavir, midazolam, lorazepam, flumazenil, dexmedetomidine, ketamine, fentanyl, hydromorphone, butorphanol, naloxone, insulin, fluticasone, ciclesonide, budesonide, dupilumab, mometasone, albuterol, reslizumab, zileuton, mepolizumab, omalizumab, and haloperidol.

25. The composition of claim 1, wherein the weight-to-weight ratio of GO-PEI particles to microbial antigen ranging from 10:1 to 1:10.

26. The composition of claim 1, further comprising an adjuvant.

27. The composition of claim 26, wherein the adjuvant comprises CpG oligonucleotide (CpG ODN).

28. The composition of claim 26, wherein the weight-to-weight ratio of GO-PEI particles to microbial antigen to adjuvant comprises 10:5:2.5 or 10:5:1.

29. The composition of claim 1, wherein the combination of a functionalized GO nanoparticle, microbial antigen with or without an adjuvant has a diameter of about 50 to about 300 nm.

30. The composition of claim 1, wherein the composition has a tunable zeta potential.

31. A vaccine comprising the composition of claim 1.

32. A method of inhibiting a microbial infection, treating a microbial infection, inducing an immune response in a subject to a microbial antigen, vaccine, or pharmaceutical antigen, or delivering an antigen, vaccine, and/or pharmaceutical agent to a mucosal surface of a subject comprising administering to a subject at risk of being infected with a microbe or infected with a microbe, the vaccine of claim 31.

33. (canceled)

34. (canceled)

35. (canceled)

36. The method of inducing an immune response of claim 32, wherein the immune response comprises production of IgA antibody, IgG antibody, IL-6 cytokine production, and/or TNF-α production.

37. (canceled)

38. (canceled)

39. A method of making a vaccine to a microbial antigen comprising
a. obtaining a graphene oxide (GO) powder;
b. sonicating GO flakes in an ice bath until the GO particles are less than 500 nm in diameter using tip ultrasonication;
c. adding polyethyleneimine (PEI) and sonicating;
d. activating the GO-PEI with EDC; and
e. adding the microbial antigen to the activated GO-PEI particles at a GO-PEI to Ag weight-to-weight ratio ranging from 10:1 to 1:10.

40. The method of making a vaccine to a microbial antigen of claim 39, wherein the sonication continues until the GO particles in step b are less than 200 nm in diameter.

41. (canceled)

42. The method of making a vaccine to a microbial antigen of claim 39, further comprising PEGylating the GO-PEI particles.

43. The method of making a vaccine to a microbial antigen of claim 39, further comprising co-loading an adjuvant with the microbial antigen onto the GO-PEI nanoparticles.

44. The method of making a vaccine to a microbial antigen of claim 43, wherein the weight-to-weight ratio of GO-PEI particles to microbial antigen to adjuvant comprises 10:5:2.5 or 10:5:1.
